(19) United States
(12) Patent Application Publication
(10) Pub. No.: US 2025/0329424 A1
(43) Pub. Date: Oct. 23, 2025

(54) Oral Health Care Assessment Devices And Methods

(71) Applicant: Colgate-Palmolive Company, New York, NY (US)

(72) Inventors: Indrani BANERJEE, Basking Ridge, NJ (US); Donghui WU, Bridge water, NJ (US); Keith Matthew CARROLL, Somerset, NJ (US)

(73) Assignee: Colgate-Palmolive Company, New York, NY (US)

(21) Appl. No.: 19/182,826

(22) Filed: Apr. 18, 2025

Related U.S. Application Data
(60) Provisional application No. 63/636,148, filed on Apr. 19, 2024.

(51) Int. Cl. G16H 10/20; G16H 10/60; G16H 20/00; G16H 50/30; G16H 80/00

(52) U.S. Cl. CPC G16H 10/20; G16H 10/60; G16H 20/00; G16H 50/30; G16H 80/00

(57) ABSTRACT

A computer-implemented technique may comprise receiving, for each of a dental user subject of a plurality of dental user subjects, survey result information respectively relating to an executed survey by each dental user subject. At least one machine-learning model may be selected based at least on the survey result information. Techniques may comprise inputting, into the at least one machine-learning model, the received survey result information. The at least one machine-learning model may determine at least one dental condition score for each dental user subject based, at least in part, on the survey result information. Techniques may comprise producing the at least one dental condition score for each dental user patient in a visually interpretable form, and/or an electronic form. The at least one machine-learning model may be calibrated, at least in part, with one or more dental treatment codes from dental study subjects.

### BACKGROUND

Various personal care devices, such as a toothbrush, razor, water pick, etc., may be used to clean the one or more body parts (e.g., teeth/dentition) in an target area (e.g., oral cavity, legs, arm pits, etc.), for example by removing plaque and/or debris from the tooth/dentition surfaces. Toothbrushes can be used for numerous oral health purposes. For example, toothbrushes can be used for whitening teeth, killing bacteria within the oral cavity/mouth, detecting the presence of bacteria within the mouth, increasing blood circulation for gum therapy, and/or reducing the pain from gum inflammation.

Dental tissue issues such as, for example, periodontal disease and dental caries (e.g., tooth cavities) are often caused by certain bacterial species in the mouth that interact with proteins present in saliva to form a film, known as plaque, that coats the teeth. If such build-up progresses, the acid produced by the bacteria can attack the teeth resulting in tooth decay. The plaque also may attack the soft gum tissue of the mouth leading to gingivitis, or periodontitis, which may affect much, if not all, of the soft tissue and bone supporting the teeth.

Gingivitis is a form of gum disease that causes material irritation of the gingiva. The gingiva is a part of the oral mucosa that covers the alveolar bone and tooth root to a level just coronal to the cement-enamel junction. In other words, the gingiva is the area/section of the gum around the base of a tooth or around the teeth. Indications of gingivitis can include swollen gums/gingiva, red gums/gingiva, bleeding gums/gingiva, receding gums/gingiva, tooth loss, and/or halitosis.

Dental caries may be a relatively common disease, especially in some geographies and cultures. Dental caries is a chronic infectious disease that may be caused by tooth-adherent cariogenic bacteria that metabolize into sugars to produce acid. Over time, dental caries can demineralize and compromise tooth structure.

### BRIEF SUMMARY

One or more techniques described herein may include computer-implemented methods and/or devices performing same. One or more methods may comprise receiving, for each dental user subject of a plurality of dental user subjects, survey result information respectively relating to an executed survey by each dental user subject. One or more methods may comprise selecting at least one machine-learning model based at least on the survey result information. One or more methods may comprise inputting, into the at least one machine-learning model, the received survey result information. One or more methods may comprise determining, by the at least one machine-learning model, at least one dental condition score for each dental user subject based, at least in part, on the survey result information. One or more methods may comprise producing the at least one dental condition score for each dental user patient in a visually interpretable form, and/or an electronic form. One or more of the method elements may be performed by one or more processing devices.

One or more techniques described herein may include computer-implemented methods and/or devices performing same. One or more methods may comprise receiving, for each dental study subject of a plurality of dental study subjects, one or more dental treatment codes respectively relating to a dental history of each dental study subject. One or more methods may comprise receiving, for each dental study subject of the plurality of dental study subjects, study survey result information respectively relating to an executed study survey by each dental study subject. At least one machine learning model may be selected based on the one or more dental treatment codes, and/or the study survey result information. One or more methods may comprise associating, by the at least one machine learning model, the one or more dental treatment codes and the survey result information. One or more methods may comprise calibrating the at least one machine-learning algorithm based, at least in part, on the associated one or more dental treatment codes and the received study survey result information. The at least one machine-learning model may determine a calibration dental condition score. One or more methods may comprise producing the at least one calibration dental condition score for each dental study subject in a visually interpretable form, and/or an electronic form. One or more of the method elements may be performed by one or more processing devices.

One or more techniques described herein may include computer-implemented methods and/or devices performing same. One or more methods may comprise receiving, for each dental study subject of the plurality of dental study subjects, study survey result information respectively relating to an executed study survey by each dental study subject. One or more methods may comprise receiving, for one or more dental study subject of the plurality of dental study subjects, one or more dental records comprising dental records for treatment of dental caries and/or periodontitis. One or more methods may comprise selecting at least one machine learning model based on the study survey result information, and/or the one or more dental records. One or more methods may comprise associating, by the at least one machine learning model, the one or more dental records and the survey result information. One or methods may comprise calibrating the at least one machine-learning algorithm based, at least in part, on the associated one or more dental records and the received study survey result information. One or methods may comprise determining, by the at least one machine-learning model, a calibration dental condition score. One or more methods may comprise producing the at least one calibration dental condition score for each dental study subject in a visually interpretable form, and/or an electronic form. One or more of the method elements may be performed by one or more processing devices.

### BRIEF DESCRIPTION OF THE DRAWINGS

The present disclosure will become more fully understood from the detailed description and the accompanying drawings, wherein:

FIG. 1 illustrates an example chart of responses to study surveys from over three thousand dental study subjects.

FIG. 2 illustrates an example chart of responses to study surveys from over three thousand dental study subjects.

FIG. 3A is an example of two (2) Receiver Operating Characteristic-Area Under the Curve (ROC-AUC) of machine-learning (ML) model parameters for dental caries based on replies to most/all dental survey questions.

FIG. 3B is an example of two (2) Receiver Operating Characteristic-Area Under the Curve (ROC-AUC) of machine-learning (ML) model parameters for dental caries based on replies to most/all dental survey questions.

FIG. 4A is an example of two (2) Receiver Operating Characteristic-Area Under the Curve (ROC-AUC) of machine-learning (ML) model parameters for dental caries based on replies to selected dental survey questions.

FIG. 4B is an example of two (2) Receiver Operating Characteristic-Area Under the Curve (ROC-AUC) of machine-learning (ML) model parameters for dental caries based on replies to selected dental survey questions.

FIG. 4C is an example of Receiver Operating Characteristic-Area Under the Curve (ROC-AUC) values of machine-learning (ML) model parameters for dental caries.

FIG. 5A is an example of two (2) Receiver Operating Characteristic-Area Under the Curve (ROC-AUC) of machine-learning (ML) model parameters for periodontitis based on replies to most/all dental survey questions.

FIG. 5B is an example of two (2) Receiver Operating Characteristic-Area Under the Curve (ROC-AUC) of machine-learning (ML) model parameters for periodontitis based on replies to most/all dental survey questions.

FIG. 6A is an example of two (2) Receiver Operating Characteristic-Area Under the Curve (ROC-AUC) of machine-learning (ML) model parameters for periodontitis based on replies to selected dental survey questions.

FIG. 6B is an example of two (2) Receiver Operating Characteristic-Area Under the Curve (ROC-AUC) of machine-learning (ML) model parameters for periodontitis based on replies to selected dental survey questions.

FIG. 6C is an example of Receiver Operating Characteristic-Area Under the Curve (ROC-AUC) values of machine-learning (ML) model parameters for periodontitis.

FIG. 6D is an example of two (2) Receiver Operating Characteristic-Area Under the Curve (ROC-AUC) of machine-learning (ML) model parameters for periodontitis including past three(s) years of dental history.

FIG. 6E is an example of two (2) Receiver Operating Characteristic-Area Under the Curve (ROC-AUC) of machine-learning (ML) model parameters for periodontitis including past three(s) years of dental history.

FIG. 7 illustrates an example diagram of an oral health assessment user interface in which a dental user subject initiates the survey process.

FIG. 8 illustrates an example diagram of an oral health assessment user interface in which a dental user subject replies to a survey question and receives a comment on the reply.

FIG. 9 illustrates an example diagram of an oral health assessment user interface in which a dental user subject replies to a survey question and receives a comment on the reply.

FIG. 10 illustrates an example diagram of an oral health assessment user interface in which a dental user subject replies to a survey question and receives a comment on the reply.

FIG. 11 illustrates an example diagram of an oral health assessment user interface in which a dental user subject replies to a survey question and receives a comment on the reply.

FIG. 12 illustrates an example diagram of an oral health assessment user interface in which a dental user subject replies to a survey question and receives a comment on the reply.

FIG. 13 illustrates an example diagram of an oral health assessment user interface illustrating a comment for a reply to two possible survey answers.

FIG. 14 illustrates an example diagram of an oral health assessment user interface in which a dental user subject replies to a survey question and receives a comment on the reply.

FIG. 15 illustrates an example diagram of an oral health assessment user interface in which a dental user subject replies to a survey question and receives a comment on the reply.

FIG. 16 illustrates an example diagram of an oral health assessment user interface in which a dental user subject replies to a survey question and receives a comment on the reply.

FIG. 17 illustrates an example diagram of an oral health assessment user interface in which a dental user subject replies to a survey question and receives a comment on the reply.

FIG. 18 illustrates an example diagram of an oral health assessment user interface in which a dental user subject replies to a survey question and receives a comment on the reply.

FIG. 19 illustrates an example diagram of an oral health assessment user interface in which a dental user subject replies to a survey question and receives a comment on the reply.

FIG. 20 illustrates an example diagram of an oral health assessment user interface in which a dental user subject replies to a survey question and receives a comment on the reply.

FIG. 21 illustrates an example diagram of an oral health assessment user interface illustrating a comment for a reply to two possible survey answers.

FIG. 22 illustrates an example diagram of an oral health assessment user interface illustrating a comment for a reply to four possible survey answers.

FIG. 23 illustrates an example diagram of an oral health assessment user interface in which a dental user subject is presented with at least three survey questions.

FIG. 24 illustrates an example diagram of an oral health assessment user interface in which a dental user subject is presented with at least three survey questions.

FIG. 25 illustrates an example diagram of an oral health assessment user interface in which a dental user subject is presented with a selection to produce one or more dental condition scores.

FIG. 26 illustrates an example diagram of an oral health assessment user interface in which a dental user subject is presented with two dental condition scores, one or more personalized recommendations, and links to information/literature regarding oral health.

FIG. 27A illustrates an example diagram of an oral health assessment user interface in which a dental user subject may be presented with at least two dental condition scores, one or more personalized recommendations, and links to information/literature regarding oral health.

FIG. 27B illustrates an example diagram of an oral health assessment that a dental user subject may be presented with that includes a vertical bar chart indicator with one or more dental condition/risk scores.

FIG. 27C illustrates an example diagram of an oral health assessment that a dental user subject may be presented with that includes a horizontal bar chart indicator with one or more dental condition/risk scores.

FIG. 27D illustrates an example diagram of an oral health assessment that a dental user subject may be presented with that includes a traffic light-type indicator with one or more dental condition/risk warning levels.

FIG. 27E illustrates an example diagram of an oral health assessment that a dental user subject may be presented with that includes a racetrack-type indicator with one or more dental condition/risk warning levels.

FIG. 27F illustrates an example diagram of an oral health assessment that a dental user subject may be presented with that includes a spider chart-type indicator with one or more dental condition/risk warning levels.

FIG. 27G illustrates an example diagram of an oral health assessment that a dental user subject may be presented with that includes one or more fuel gauge chart indicators with one or more dental condition/risk warning levels.

FIG. 28 illustrates three questions of an example oral health survey that may be presented to dental study subjects and/or dental user subjects.

FIG. 29 illustrates two questions of an example oral health survey that may be presented to dental study subjects and/or dental user subjects.

FIG. 30 illustrates three questions of an example oral health survey that may be presented to dental study subjects and/or dental user subjects.

FIG. 31 illustrates three questions of an example oral health survey that may be presented to dental study subjects and/or dental user subjects.

FIG. 32 illustrates three questions of an example oral health survey that may be presented to dental study subjects and/or dental user subjects.

FIG. 33 illustrates three questions of an example oral health survey that may be presented to dental study subjects and/or dental user subjects.

FIG. 34 illustrates two questions of an example oral health survey that may be presented to dental study subjects and/or dental user subjects.

FIG. 35 illustrates three questions of an example oral health survey that may be presented to dental study subjects and/or dental user subjects.

FIG. 36 illustrates two questions of an example oral health survey that may be presented to dental study subjects and/or dental user subjects.

FIG. 37 is a block diagram of a hardware configuration of an example device that may control and/or implement one or more parts of the disclosed oral health assessment.

FIG. 38 is a flowchart of an example technique for determining at least one dental condition score for a dental user patient based on an executed survey.

FIG. 39 is a flowchart of an example technique for calibration of one or more machine-learning algorithms to produce a dental calibration score for a dental study subject.

The drawings represent one or more aspects of the disclosure and do not limit the scope of invention.

### DETAILED DESCRIPTION

The following description of the preferred embodiment(s) is merely exemplary in nature and is in no way intended to limit the invention or inventions. The description of illustrative embodiments is intended to be read in connection with the accompanying drawings, which are to be considered part of the entire written description. In the description of the exemplary embodiments disclosed herein, any reference to direction or orientation is merely intended for convenience of description and is not intended in any way to limit the scope of the present inventions. Relative terms such as “lower,” “upper,” “horizontal,” “vertical,” “above,” “below,” “up,” “down,” “left,” “right,” “top,” “bottom,” “front” and “rear” as well as derivatives thereof (e.g., “horizontally,” “downwardly,” “upwardly,” etc.) should be construed to refer to the orientation as then described or as shown in the drawing under discussion. These relative terms are for convenience of description only and do not require a particular orientation unless explicitly indicated as such. Terms such as “attached,” “affixed,” “connected,” “coupled,” “interconnected,” “secured” and other similar terms refer to a relationship wherein structures are secured or attached to one another either directly or indirectly through intervening structures, as well as both movable or rigid attachments or relationships, unless expressly described otherwise. The discussion herein describes and illustrates some possible non-limiting combinations of features that may exist alone or in other combinations of features. Furthermore, as used herein, the term “or” is to be interpreted as a logical operator that results in true whenever one or more of its operands are true. Furthermore, as used herein, the phrase “based on” is to be interpreted as meaning “based at least in part on,” and therefore is not limited to the interpretation “based entirely on.”

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

In the following description, where block diagrams or circuits are shown and described, one of skill in the art will recognize that, for the sake of clarity, not all peripheral components or circuits are shown in the figures or described in the description. For example, common components such as memory devices and power sources may not be discussed herein, as their role would be easily understood by those of ordinary skill in the art. Further, the terms “couple” and “operably couple” can refer to a direct or indirect coupling of two components of a circuit.

Features of the present inventions may be implemented in software, hardware, firmware, or combinations thereof. The computer programs described herein are not limited to any particular embodiment, and may be implemented in an operating system, application program, foreground or background processes, driver, or any combination thereof. The computer programs may be executed on a single computer or server processor or multiple computer or server processors.

Processors described herein may be any central processing unit (CPU), microprocessor, micro-controller, computational, or programmable device or circuit configured for executing computer program instructions (e.g., code). Various processors may be embodied in computer and/or server hardware of any suitable type (e.g., desktop, laptop, notebook, tablets, cellular phones, etc.) and may include all the usual ancillary components necessary to form a functional data processing device including without limitation a bus, software and data storage such as volatile and non-volatile memory, input/output devices, graphical user interfaces (GUIs), removable data storage, and wired and/or wireless communication interface devices including Wi-Fi™, Bluetooth™, LAN, cellular, satellite, etc.

Computer-executable instructions or programs (e.g., software or code) and data described herein may be programmed into and tangibly embodied in a non-transitory computer-readable medium that is accessible to and retrievable by a respective processor as described herein which configures and directs the processor to perform the desired functions and processes by executing the instructions encoded in the medium. A device embodying a programmable processor configured to such non-transitory computer-executable instructions or programs may be referred to as a “programmable device”, or “device”, and multiple programmable devices in mutual communication may be referred to as a “programmable system.” It should be noted that non-transitory “computer-readable medium” as described herein may include, without limitation, any suitable volatile or non-volatile memory including random access memory (RAM) and various types thereof, read-only memory (ROM) and various types thereof, USB flash memory, and magnetic or optical data storage devices (e.g., internal/external hard disks, floppy discs, magnetic tape CD-ROM, DVD-ROM, optical disk, ZIP™ drive, Blu-ray disk, and others), which may be written to and/or read by a processor operably connected to the medium.

In certain scenarios, the any of the subject matter disclosed herein may be embodied in the form of computer-implemented processes and apparatuses such as processor-based data processing and communication systems or computer systems for practicing those processes. The present inventions may also be embodied in the form of software or computer program code embodied in a non-transitory computer-readable storage medium, which when loaded into and executed by the data processing and communications systems or computer systems, the computer program code segments configure the processor to create specific logic circuits configured for implementing the processes.

The paradigm of healthcare has been undergoing a significant shift from a primary focus on treating diseases to a more proactive approach that emphasizes prevention. This transition recognizes the importance of early detection and intervention in mitigating the progression of diseases and improving patient outcomes. Routine medical exams play a useful (e.g., important, crucial, etc.) role in this preventative strategy, serving as a cornerstone for maintaining good health. Routine medical exams may include asking patients/dental subjects (e.g., key) questions related to their background, lifestyle, and/or medical history. One or more of these factors, perhaps along with the results from the medical exams allow healthcare providers to establish a baseline of an individual's health, track changes over time, and/or identify risk factors and/or early signs of illness that may not yet produce symptoms. By catching potential health issues early, interventions can be less invasive, more effective, and/or less costly.

Oral health is important for overall health and/or wellbeing. Perhaps along with routine dental checkups, dentists often use risk assessment questionnaires to assess oral health conditions like caries, gum health, etc. A self-assessment survey to predict oral health scores and/or identify potential risks to oral health conditions such as caries, gum disease, enamel erosion, sensitivity, dry mouth, oral malodor, and/or mouth aging, etc., may empower people/dental subjects and/or raise awareness and/or enable them to use appropriate measures on time and/or help in maintaining good oral health.

Health care has in many ways shifted from a focus on fixing and/or healing health issues to predicting and preventing health issues/problems. Oral health is important for overall health and wellbeing. Self-implemented and timely oral health assessments may be a useful (e.g., important) tool, and may serve as a useful (e.g., critical) way to monitor oral health and/or as a warning to take appropriate precautions. Dentists may use risk assessment questionnaires to assess oral health risks for caries, gum disease, other oral health conditions, etc. Techniques described herein include a consolidated self-assessment survey and machine-learning models to predict and/or identify potential risks to caries, gum disease, bad breath, dry mouth, mouth aging, enamel erosion, and/or sensitivity based, at least in part, on a subject's (e.g., dental user subject) response to the survey. This may raise oral health awareness and/or may empower dental user subjects to take better care of their oral health. This may enable dental user subjects to use appropriate measures (e.g., in a timely fashion) thus helping in maintaining good oral health. Based on the dental condition scores provided by the oral health assessments, one or more of personalized learning, lifestyle recommendations, and/or product recommendations can be provided.

One or more surveys have been developed including one or more useful questions that impact oral health based on various literatures and American Dental Association (ADA) recommendations. The survey questions may be related to oral care routines, lifestyle and habits, oral and systemic health history, and/or family history, etc.

For machine-learning training and/or calibration, among other scenarios, one or more study surveys were provided to a plurality of dental study subjects (e.g., patients) of various dental practices in the USA. Over three thousand executed dental study surveys were collected. The one or more dental treatment codes of these respondents were collected, for example up to at least the past three years from the date of the executed dental study survey. For each subject, the one or more treatment codes were obtained and/or matched/associated with the study survey responses to the survey for that subject. The one or more treatment codes were used to determine whether the dental study subject respondents had a history of caries and/or gum diseases based on presence of restorative and periodontal codes respectively, among other oral health conditions.

In one or more scenarios, the study survey responses may be assigned numerical values for input to one or more machine-learning algorithms, among other reasons, as described herein. For questions with a binary selection of “yes” and “no”, the answers may be assigned numerical values of 1 and 0, respectively, for example. For single select categorical questions, the answer choices may be assigned numbers from 0 to n based on the choices (e.g., the nth choice may be assigned value n, etc.). For example, in the case of the question “How often do you snack on sugary food?”, the answer choices of “never”, “rarely”, “occasionally”, “frequently”, “daily”, or “multiple times during the day” were assigned values from 0 to 5, respectively. In case of categorical questions with multiple selections, each selection may be given a value of either 1 or 0, perhaps for example based on whether they were selected or not. For example, in case of the question, “Do you have any relevant family history”, if a dental study subject selected family history of gum disease and cavities then those two responses may be assigned as 1 while the rest may be assigned 0.

One or more machine-learning models may be used for the oral health assessments such as Naive-Bayes, linear regression, logistic regression, decision tree, random forest, extreme gradient boosted trees (Xgboost), support vector machine (SVM), and/or K near neighbors (KNN) may be used to build oral risk assessment models for caries (e.g., teeth condition, cavities, etc.), gum disease (e.g., gum condition, periodontitis, etc.), bad breath (e.g., halitosis), erosion, mouth aging, and/or gum and or tooth sensitivity, etc.

One or more dental treatment codes of the dental study subjects/respondents were collected up to the past three years from the study survey date. The one or more dental treatment codes were used to determine whether the dental study subjects had a history of caries, gum disease, bad breath, enamel erosion, dry mouth, mouth aging, and/or sensitivity. For the caries classification models, the dental study subjects with restorative treatment codes were classified into the caries group, while the remaining were classified as non-caries/otherwise healthy. Dental study subjects with periodontal codes were classified as dental study subjects with gum disease while the remaining were classified as non-periodontal/otherwise healthy. For the continuous regression models, the total number of restorative codes and periodontal codes for a given dental study subject were used as the dependent variable and/or normalized with respect to the total number of treatment codes available for that dental study subject to generate the normalized restorative code and/or periodontal code, respectively. The accuracy and/or balanced accuracy of the machine-learning (ML) models were determined. One or more machine-learning models were built with some or all (e.g., full sets, or subsets) the questions in the study surveys as features. One or more smaller subsets of questions were selected based on questions that have/may have higher importance from the ML models and/or existing ADA recommendations.

At least some examples of dental treatment codes that may be collected are illustrated in Table 1.

TABLE 1
Example of at least some Dental Treatment Codes

| ADA treatment | | |
|---|---|---|
| codes | Description | Type |
| D2950 | Core Buildup including any | Restorative |
| | pins when required | |
| D2750 | Crown porcelain/ceramic | Restorative |
| D2740 | Crown porcelain/ceramic | Restorative |
| D4910 | Periodontal Maintenance | Periodontal |
| D4341 | Periodontal Scaling Root | Periodontal |
| | Planing Four + Teeth | |
| D1110 | Prophylaxis | Preventative |
| D1206 | Topical application of fluoride varnish | Preventative |
| D0220 | Intraoral - periapical | Diagnostic |
| | first film (Radiographs) | |
| D0120 | Periodic oral evaluation | Diagnostic |

In one or more scenarios, one or more dental treatment codes may be used in one or more classification models/algorithms. For one or more caries classification models, the respondents (e.g., dental study patients) with restorative treatment codes were classified into the caries group while the remaining were classified as a group free of caries (e.g., non-caries). For perio classification models, respondents (e.g., dental study patients) with periodontal codes were classified as people with gum disease while the remaining were classified as a group with healthy gum (e.g., non-gum disease). For the continuous regression models, the total number of restorative codes and/or periodontal codes for a given person may either be used as the dependent variable and/or normalized with respect to the total number of treatment codes available for that person to generate the dependent variable, for example.

The “Area Under the Curve” of the “Receiver Operating Characteristic” AUC-ROC ranged from 56.3-74.8% for the various ML algorithms used for the caries model with linear regression being the highest at 74.8% followed by Xgboost at 72.04% and random forest regression at 70.4%. For gum disease models the AUC-ROC ranged from 50.7-66.1% with random forest regression being the highest at 66.1% followed by Xgboost at 64.6% and linear regression at 63.1%. These may indicate robust models.

One or more ML models for caries and/or gum health were built for caries and gum health based on study survey responses and/or one or more dental treatment codes of “real world” dental study subjects. The one or more ML models exhibit reasonable ROC-AUC up to 74.8%. These ML models can be used to predict caries and/or gum disease risks based on dental user subject responses to the survey. Although examples have been provided for gum disease and caries, the techniques described herein can be applied to other oral indications, for example sensitivity, dry mouth, breath malodor, mouth aging, and/or enamel erosion, among others, etc. In one or more scenarios, one or more risk assessment models can be built using a hybrid approach which may include a combination of ML model dental condition scores and scores provided by experts (e.g., dentists, dental professionals, etc.).

In one or more scenarios, a (e.g., unique) user interface for the user survey and/or study survey may provide (e.g., impromptu) comments/recommendations to dental user subjects and/or dental study subjects, perhaps for example based, at least in part, on the replies/responses to the respective survey questions. The user interface may provide at least one personalized dental condition score and/or one or more reasons (e.g., parameters having a relatively high relevance, etc.) pertaining to the score, perhaps for example if the dental condition scores are low, among other scenarios. Such reply comments, dental condition scores, and/or reasons pertaining to the scores may help in educating people on oral health and/or may raise awareness. Personalized recommendations on oral care products, lifestyles, and/or literatures, may be provided, perhaps for example based on the at least one dental condition score.

In one or more scenarios, a dental user subject may answer the one or more survey questions. A probability (e.g., dental condition score) of caries (e.g., teeth condition/health) and/or gum disease (e.g., gum condition/health) may be determined from the one or more machine-learning models based on dental survey responses/information. In one or more scenarios, perhaps for example based on the probability, the dental user subject may be classified as low, medium, or high risk to caries and/or gum disease. Perhaps for example based on risk scoring, the dental user will get coaching, product and/or lifestyle recommendations. The dental user subject may be provided one or more factors/parameters that may have a relatively high relevance in the determination of their dental condition score(s). In one or more scenarios, a first machine-learning algorithm may be built/trained/calibrated pertaining to caries analysis. A second machine-learning algorithm may be built/trained/calibrated pertaining to periodontal analysis. One or more ML models/algorithms may be built/trained/calibrated to operate with 60-85% accuracy.

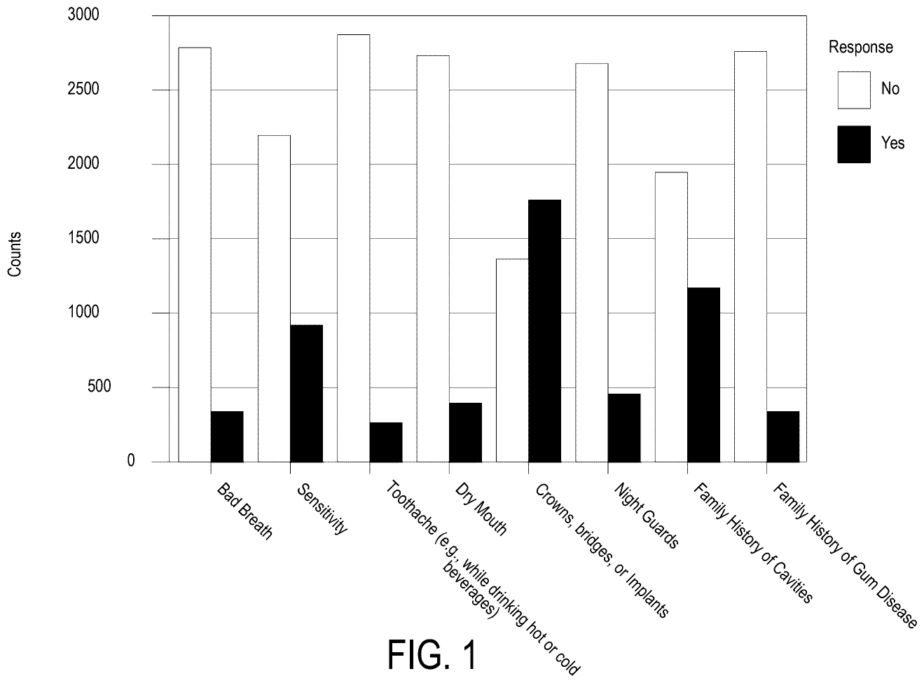

FIG. 1 illustrates an example chart of responses to study surveys from over three thousand dental study subjects.

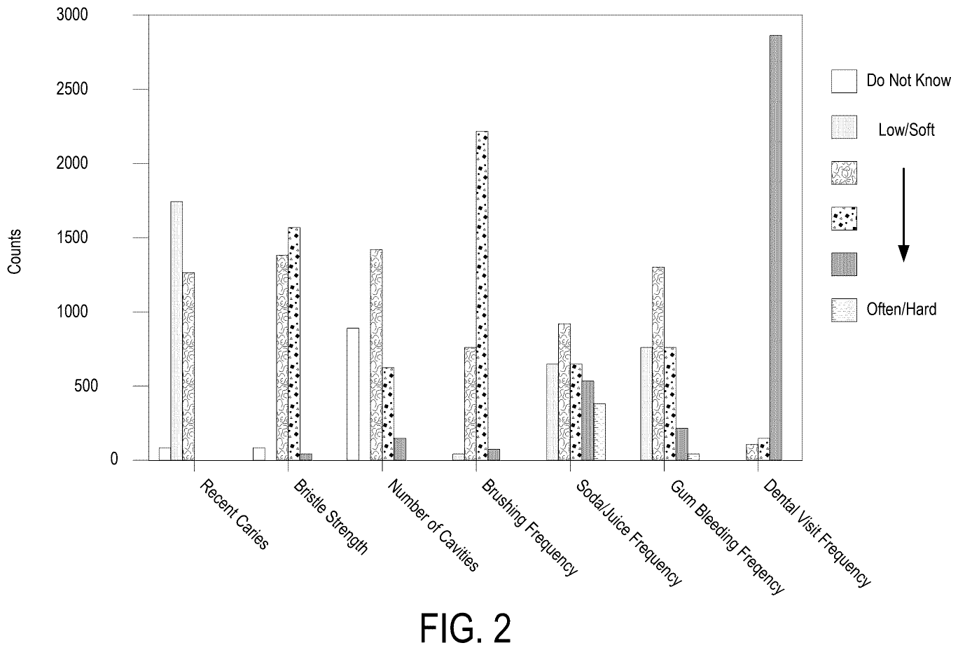

FIG. 2 illustrates an example chart of responses to study surveys from over three thousand dental study subjects.

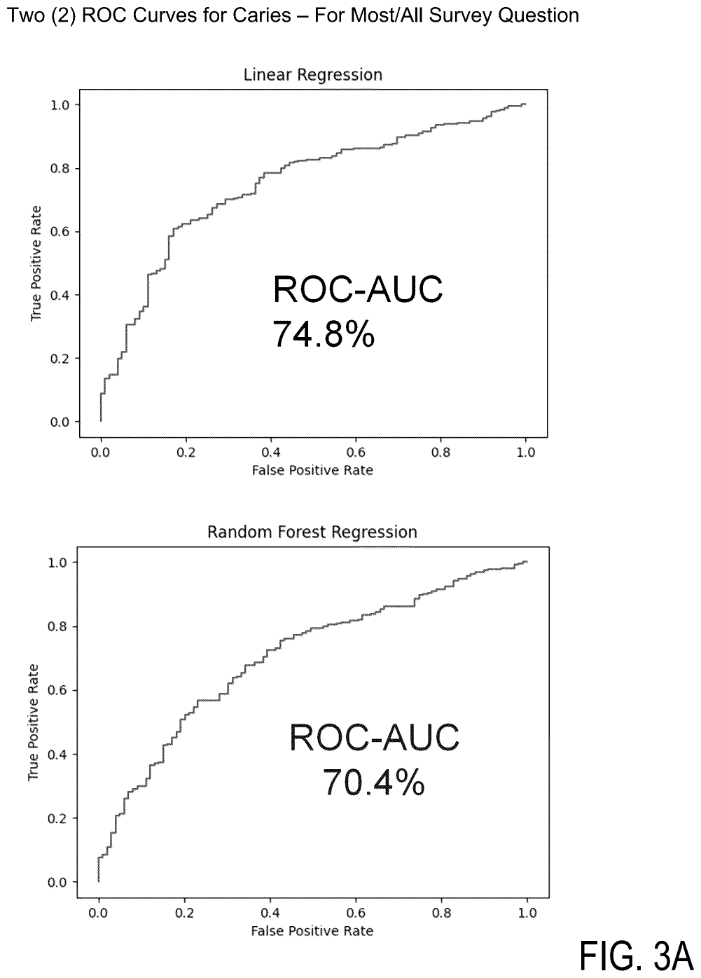

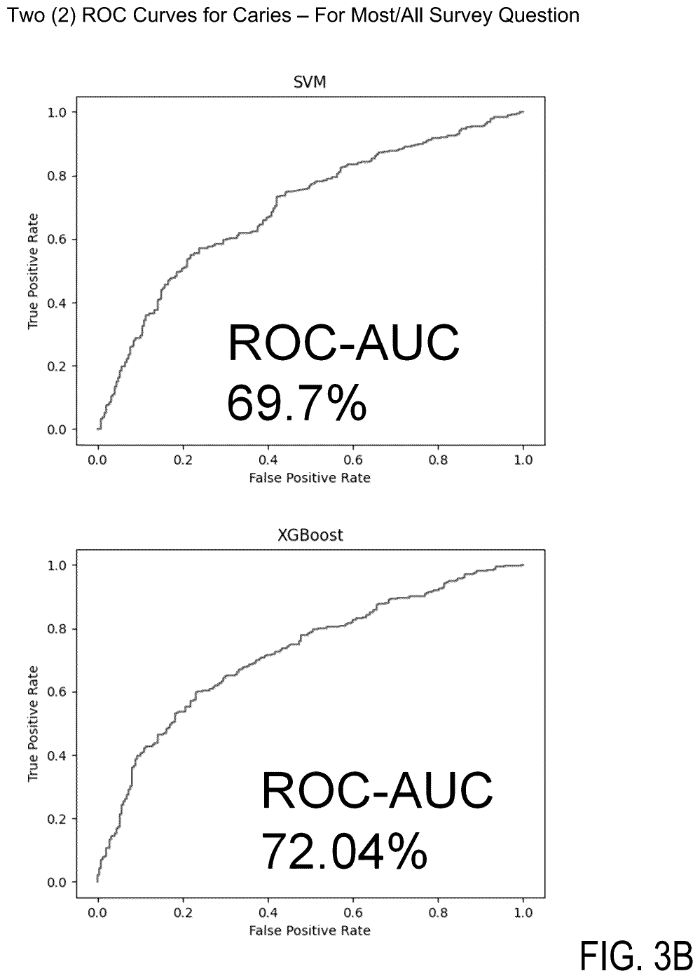

FIG. 3A is an example of two (2) Receiver Operating Characteristic-Area Under the Curve (ROC-AUC) of machine-learning (ML) model parameters for dental caries based on replies to most/all dental survey questions.

FIG. 3B is an example of two (2) Receiver Operating Characteristic-Area Under the Curve (ROC-AUC) of machine-learning (ML) model parameters for dental caries based on replies to most/all dental survey questions.

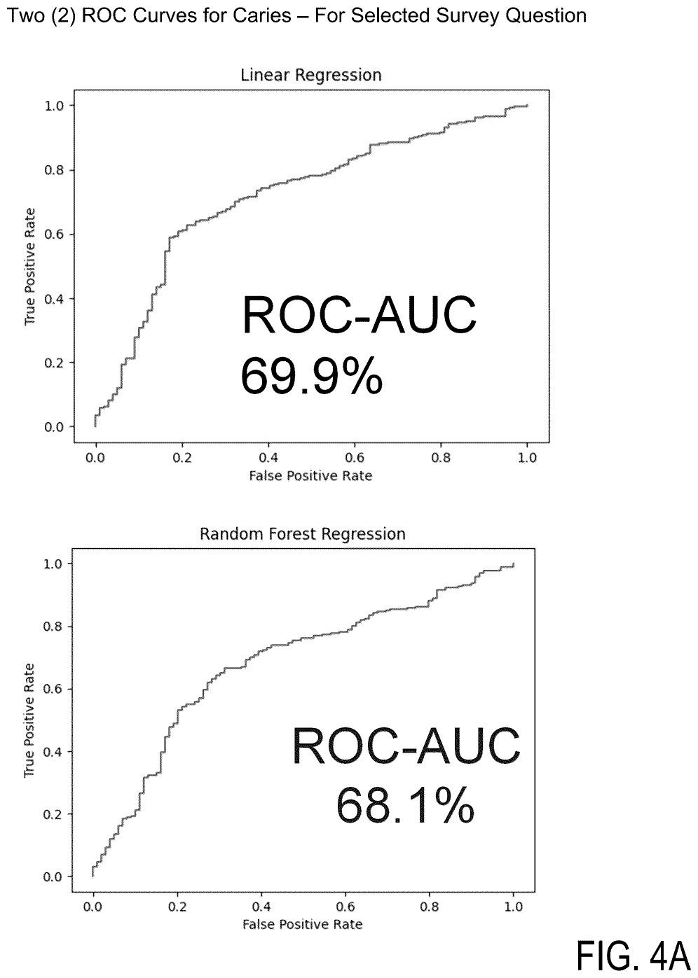

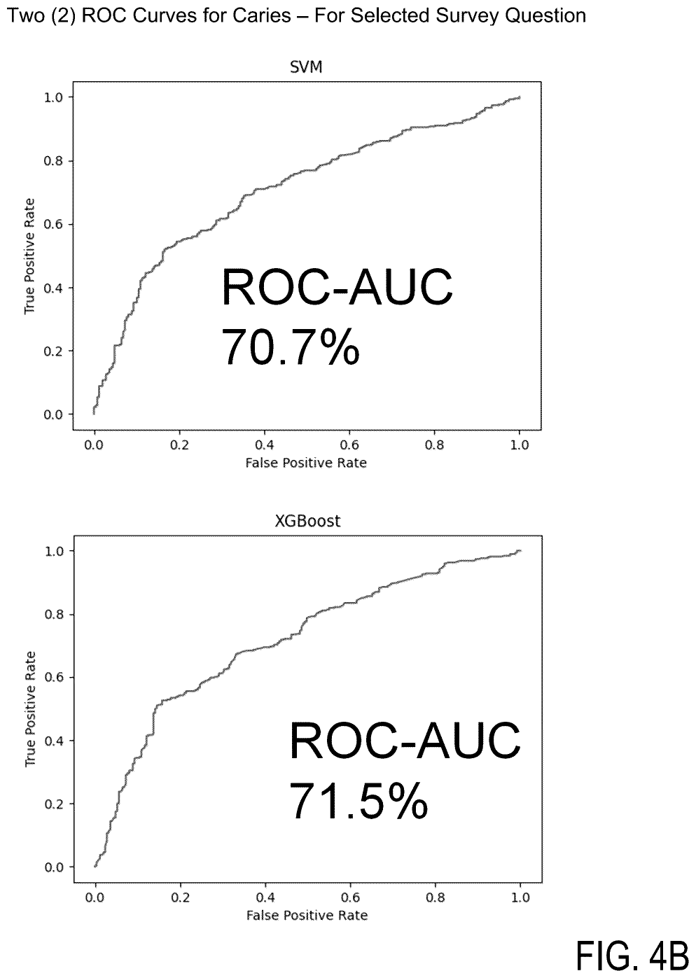

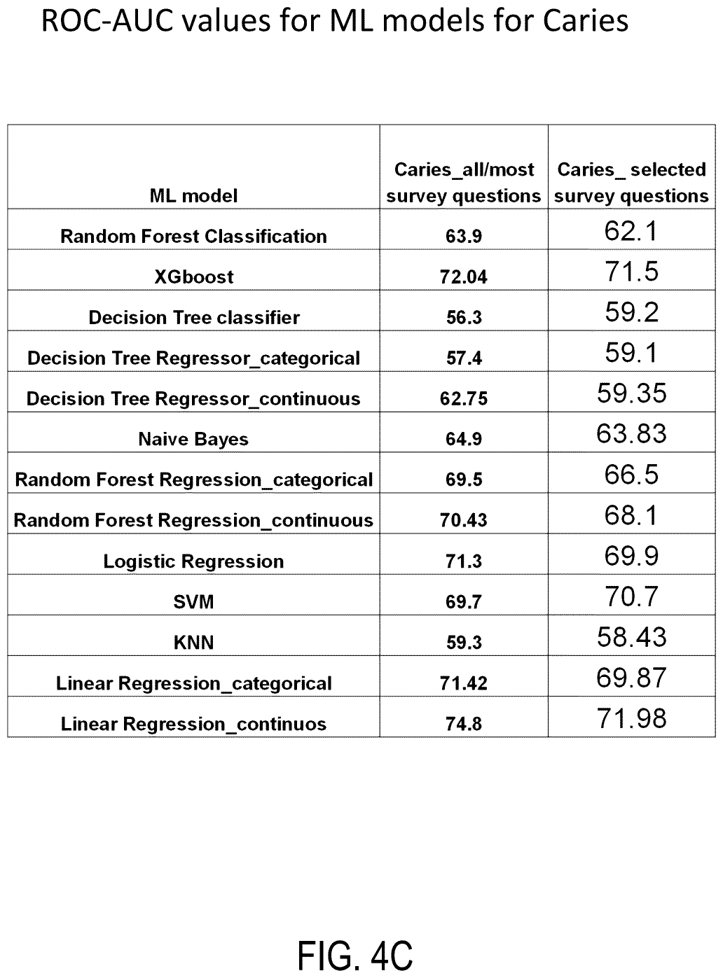

FIG. 4A is an example of two (2) Receiver Operating Characteristic-Area Under the Curve (ROC-AUC) of machine-learning (ML) model parameters for dental caries based on replies to selected dental survey questions.

FIG. 4B is an example of two (2) Receiver Operating Characteristic-Area Under the Curve (ROC-AUC) of machine-learning (ML) model parameters for dental caries based on replies to selected dental survey questions.

FIG. 4C is an example of Receiver Operating Characteristic-Area Under the Curve (ROC-AUC) values of machine-learning (ML) model parameters for dental caries.

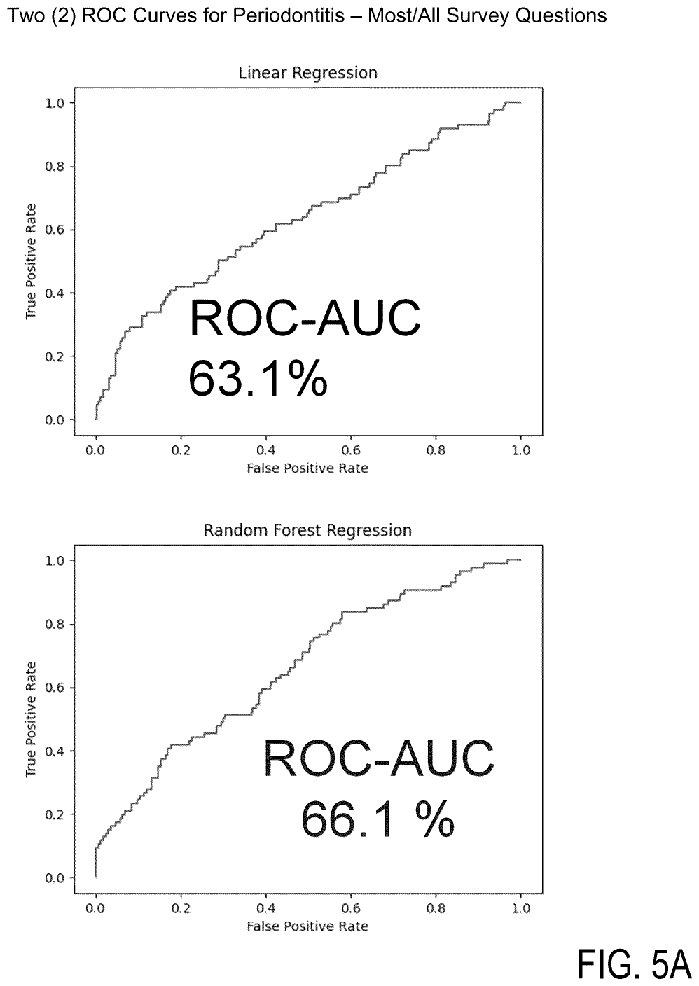

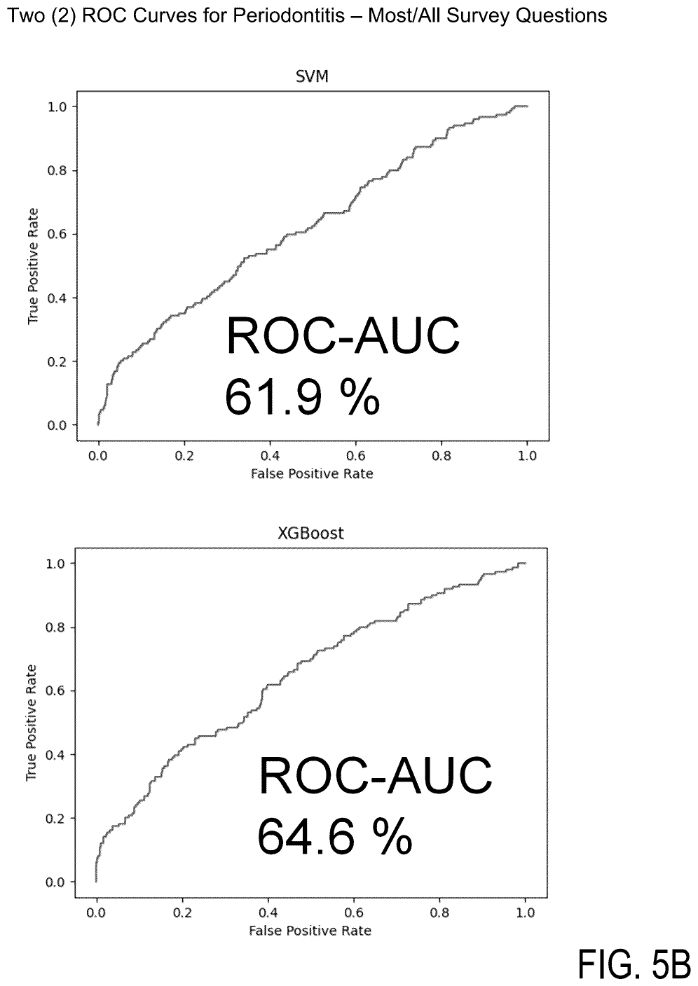

FIG. 5A is an example of two (2) Receiver Operating Characteristic-Area Under the Curve (ROC-AUC) of machine-learning (ML) model parameters for periodontitis based on replies to most/all dental survey questions.

FIG. 5B is an example of two (2) Receiver Operating Characteristic-Area Under the Curve (ROC-AUC) of machine-learning (ML) model parameters for periodontitis based on replies to most/all dental survey questions.

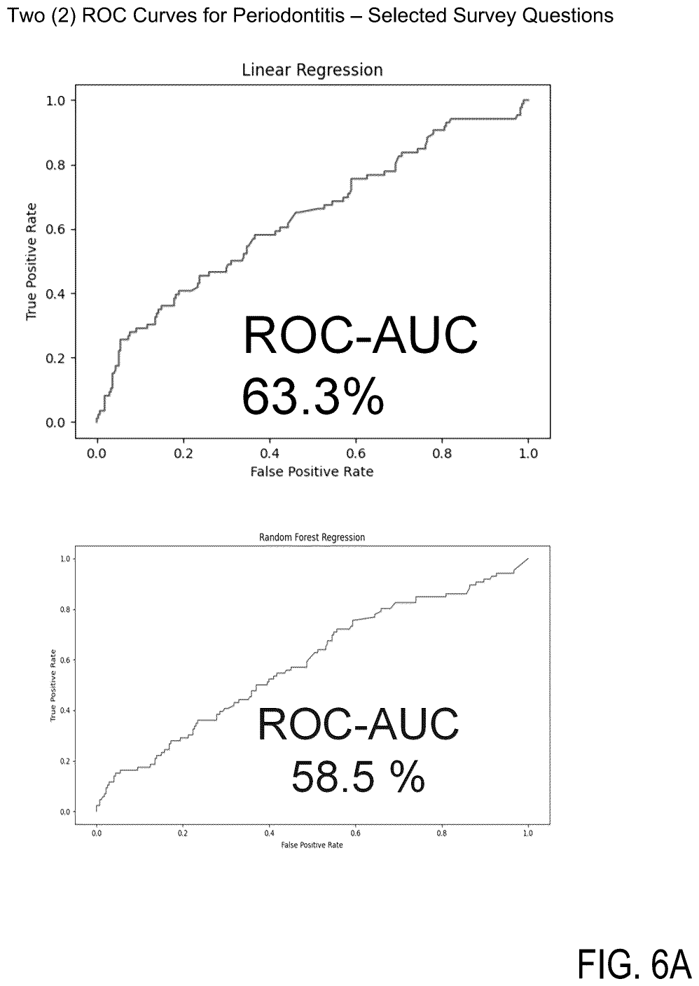

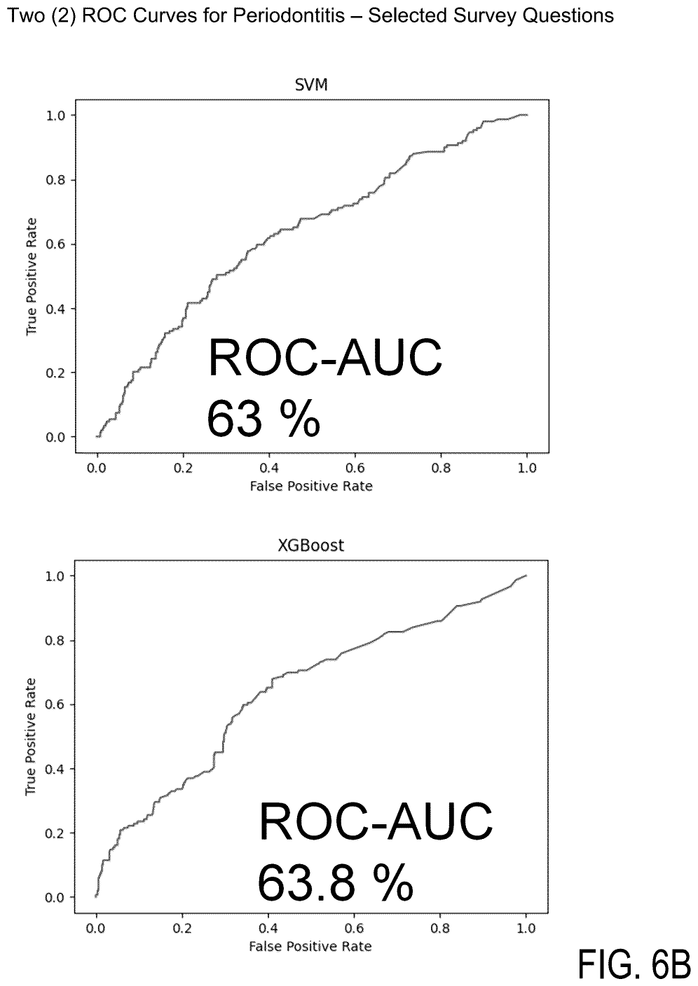

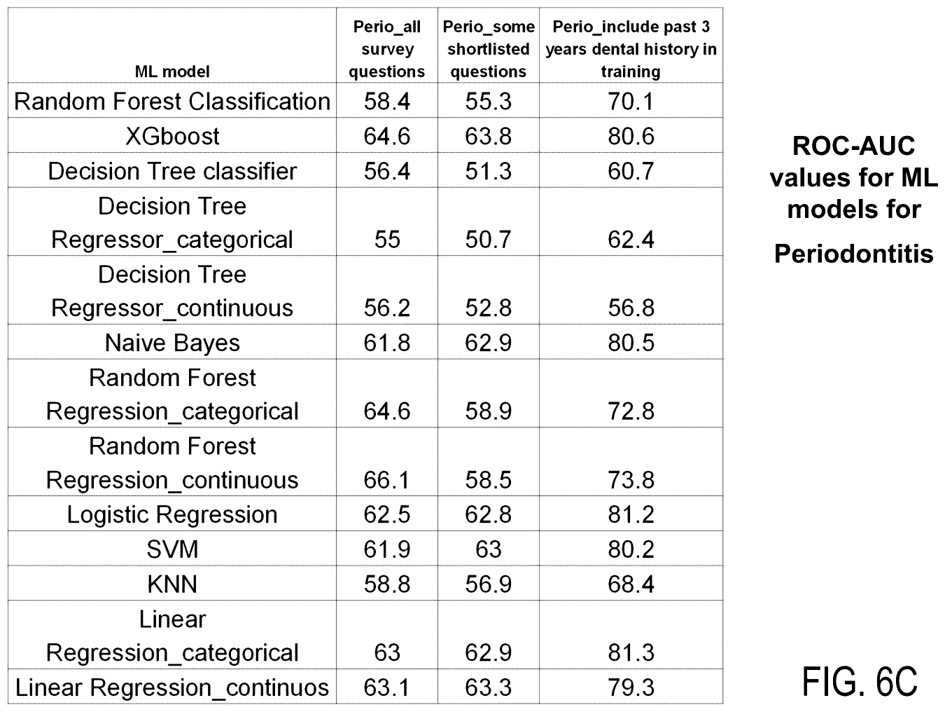

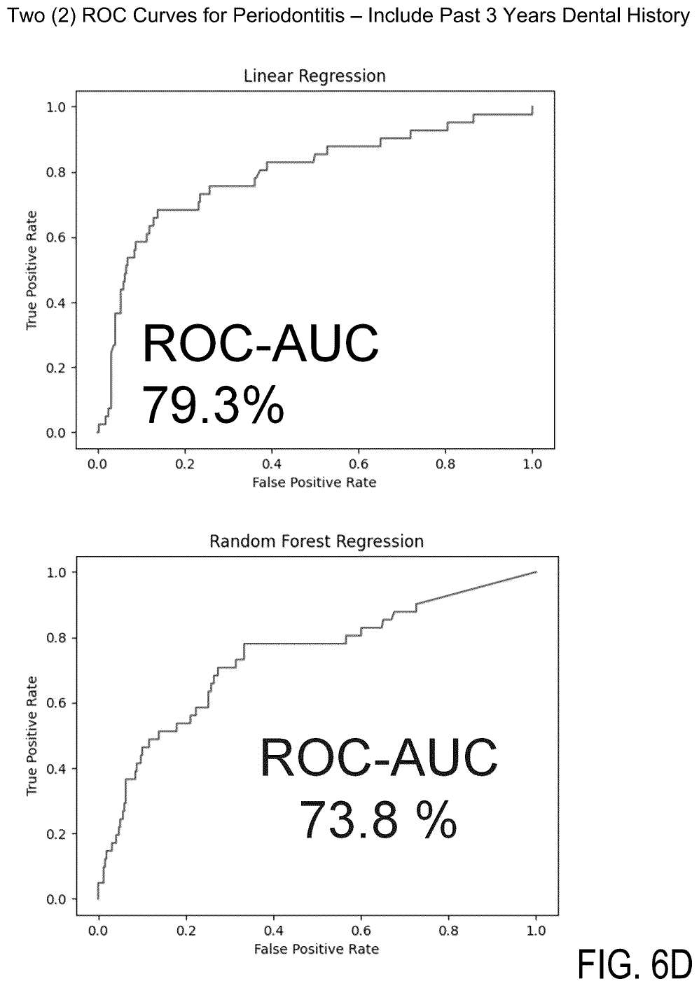

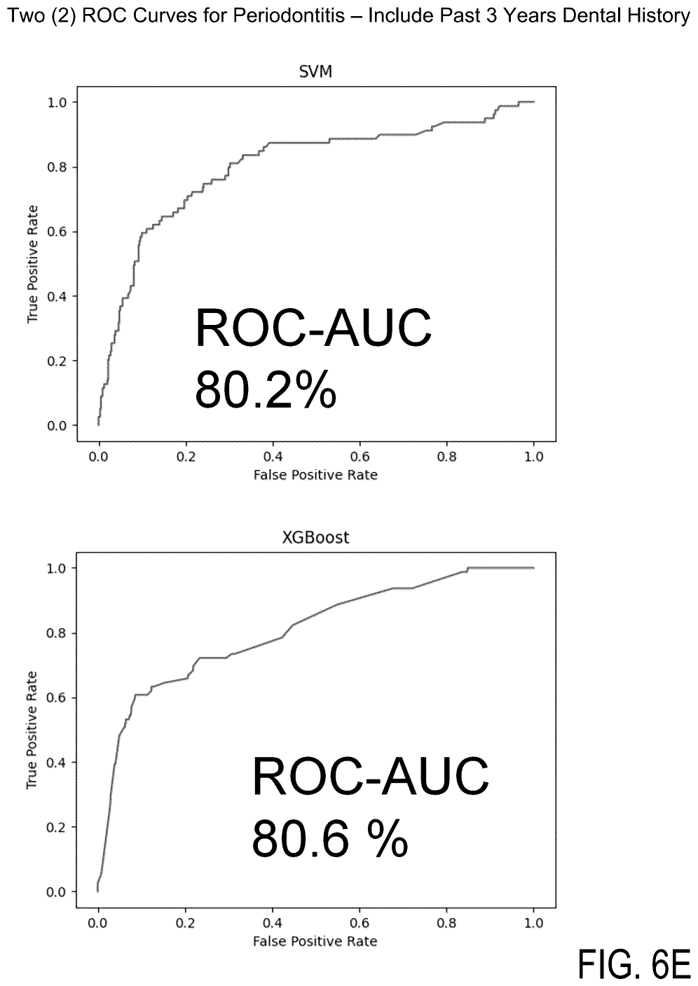

FIG. 6A is an example of two (2) Receiver Operating Characteristic-Area Under the Curve (ROC-AUC) of machine-learning (ML) model parameters for periodontitis based on replies to selected dental survey questions.

FIG. 6B is an example of two (2) Receiver Operating Characteristic-Area Under the Curve (ROC-AUC) of machine-learning (ML) model parameters for periodontitis based on replies to selected dental survey questions.

FIG. 6C is an example of Receiver Operating Characteristic-Area Under the Curve (ROC-AUC) values of machine-learning (ML) model parameters for periodontitis.

FIG. 6D is an example of two (2) Receiver Operating Characteristic-Area Under the Curve (ROC-AUC) of machine-learning (ML) model parameters for periodontitis including past three(s) years of dental history.

FIG. 6E is an example of two (2) Receiver Operating Characteristic-Area Under the Curve (ROC-AUC) of machine-learning (ML) model parameters for periodontitis including past three(s) years of dental history.

One or more models/algorithms may be built by using dental history from previous years to predict the health/disease risk for the current year. In the example shown in FIG. 6D and FIG. 6E, the self-reported response from the dental patient users for a survey question of “Ever Been Diagnosed with gum disease or referred to a periodontist?” may be replaced by a feature which represented whether the dental patient user had any periodontal treatment codes in the past three years. The feature “perio_past 3 years” may be assigned a binary value of either “1” or “0”, perhaps for example based on the presence or absence of periodontal codes for the past three years in their dental history. Dental patient users may be grouped into healthy (e.g., non-perio) and/or periodontal groups based on the absence or presence of periodontal treatment codes for the current year in which the survey was conducted. The ML algorithms may predict and/or classify dental patient users into healthy (e.g., non-perio) and perio groups with several ROC-AUCs. FIG. 6D and FIG. 6E shows the ROC-AUCs indicating clearly the highest weightage for the “perio_past 3 years.” FIG. 6D and FIG. 6E shows ROC-AUC plots for at least one oral indication but can be used for other oral indications for example caries, sensitivity, dry mouth, mouth aging, and/or enamel erosion, among other oral indications, for example.

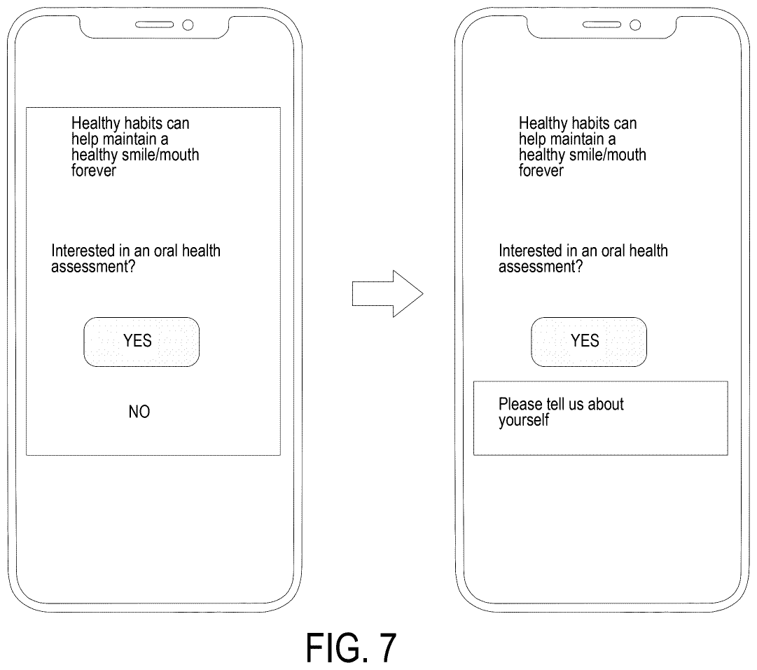

FIG. 7 illustrates an example diagram of an oral health assessment user interface in which a dental user subject initiates the survey process.

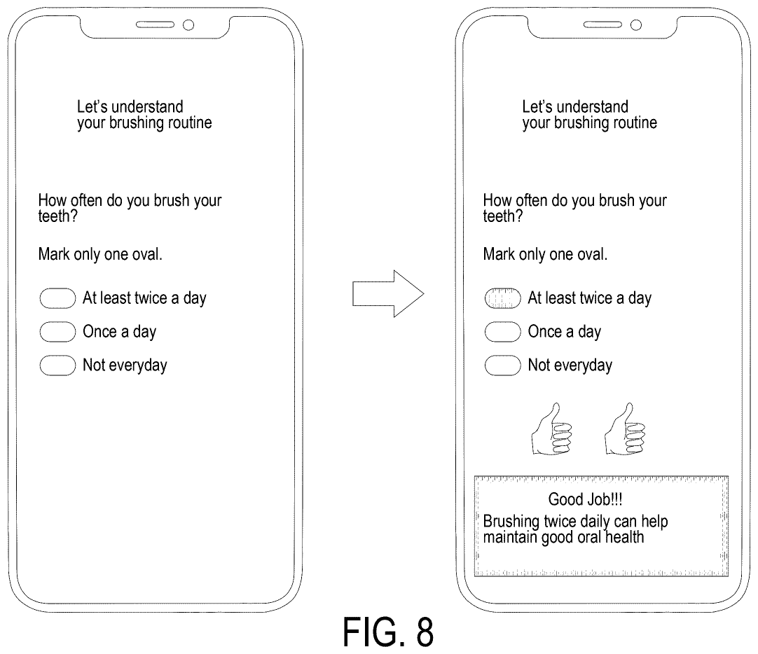

FIG. 8 illustrates an example diagram of an oral health assessment user interface in which a dental user subject replies to a survey question and receives a comment on the reply.

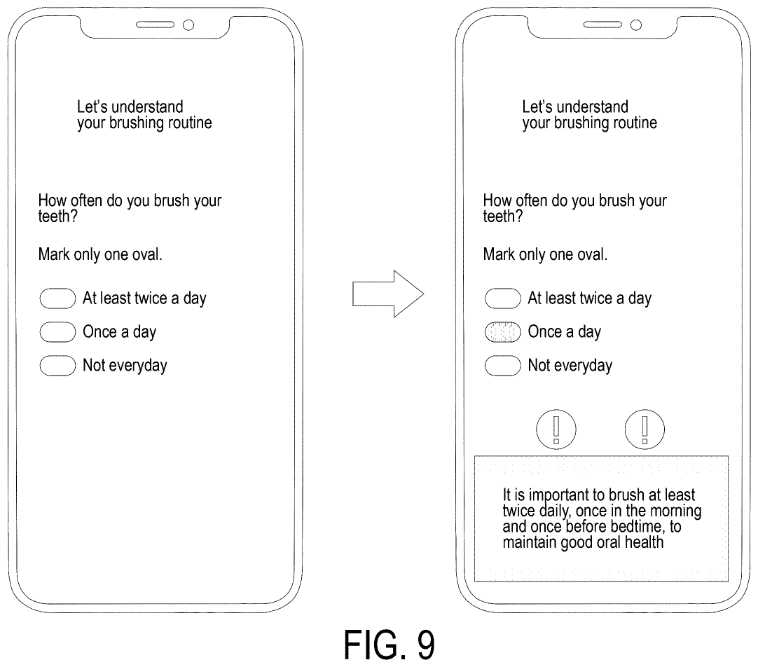

FIG. 9 illustrates an example diagram of an oral health assessment user interface in which a dental user subject replies to a survey question and receives a comment on the reply.

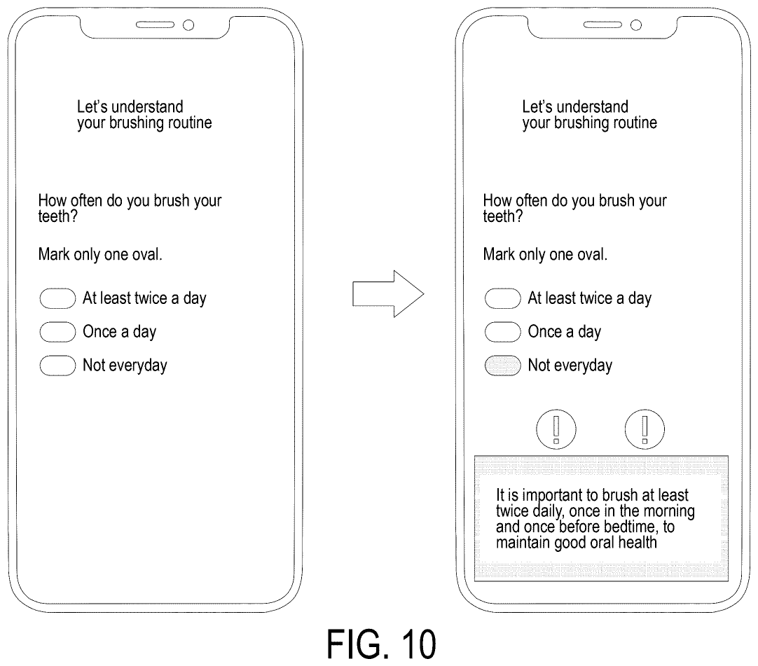

FIG. 10 illustrates an example diagram of an oral health assessment user interface in which a dental user subject replies to a survey question and receives a comment on the reply.

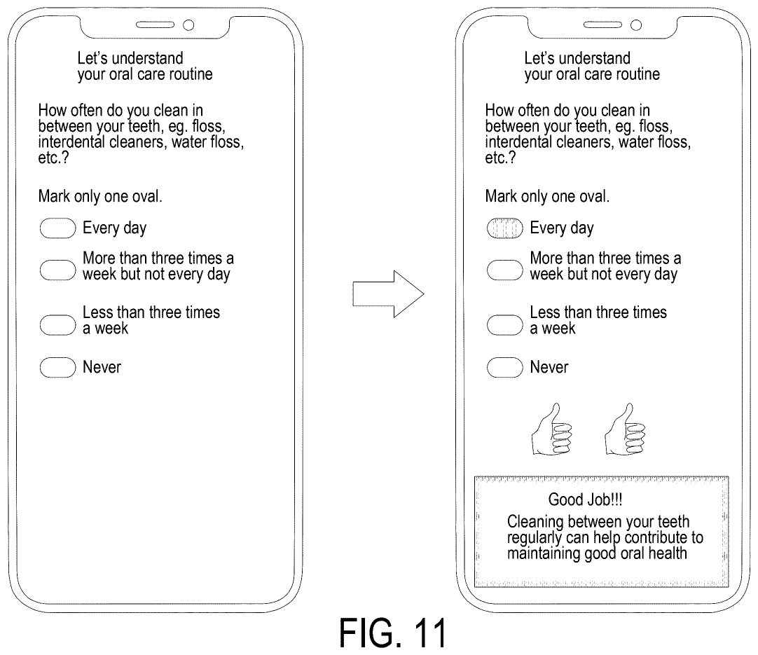

FIG. 11 illustrates an example diagram of an oral health assessment user interface in which a dental user subject replies to a survey question and receives a comment on the reply.

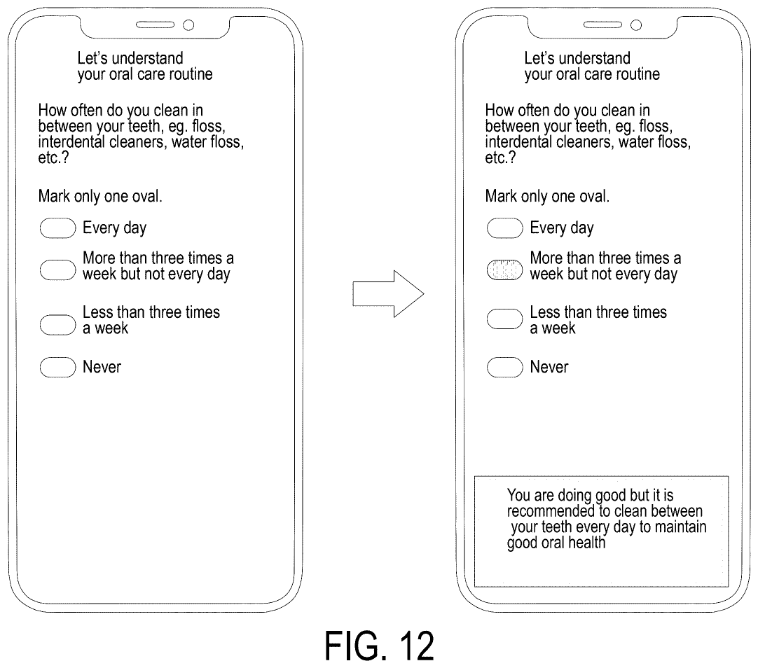

FIG. 12 illustrates an example diagram of an oral health assessment user interface in which a dental user subject replies to a survey question and receives a comment on the reply.

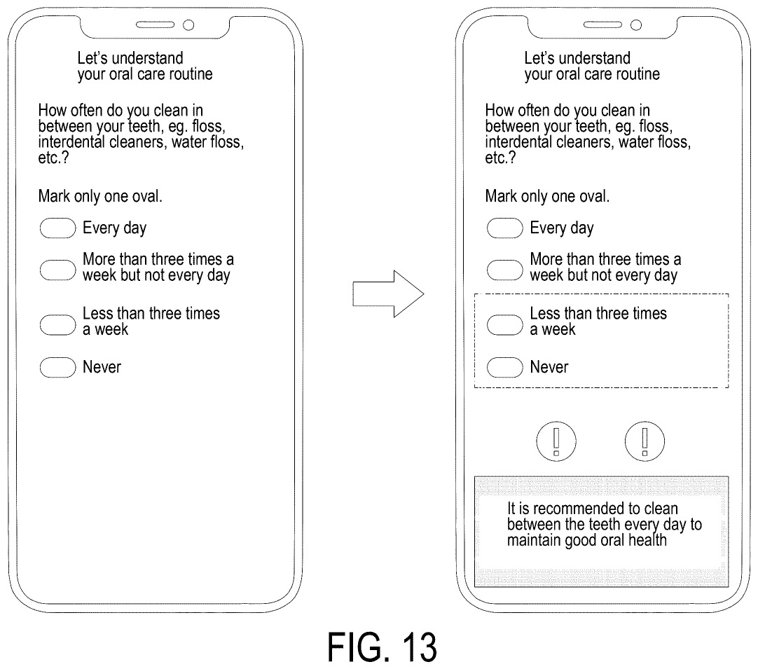

FIG. 13 illustrates an example diagram of an oral health assessment user interface illustrating a comment for a reply to two possible survey answers.

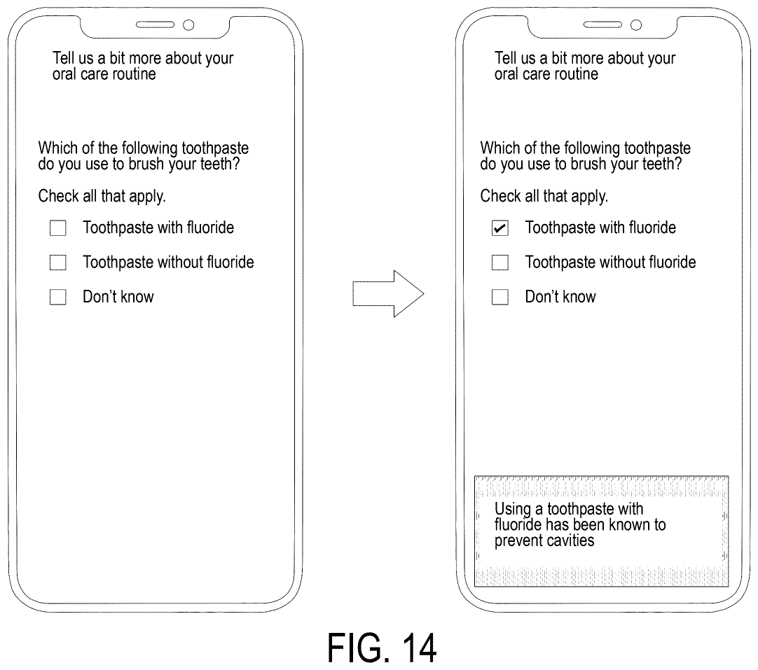

FIG. 14 illustrates an example diagram of an oral health assessment user interface in which a dental user subject replies to a survey question and receives a comment on the reply.

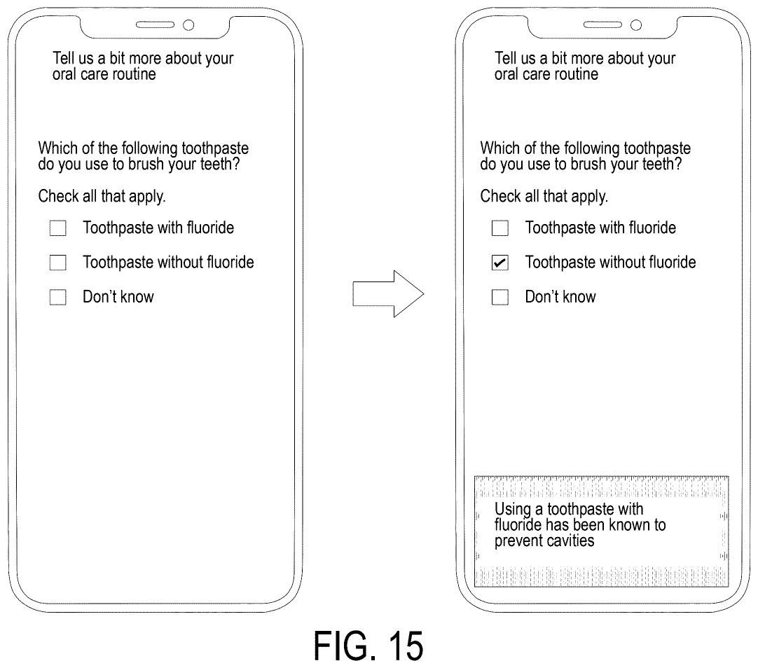

FIG. 15 illustrates an example diagram of an oral health assessment user interface in which a dental user subject replies to a survey question and receives a comment on the reply.

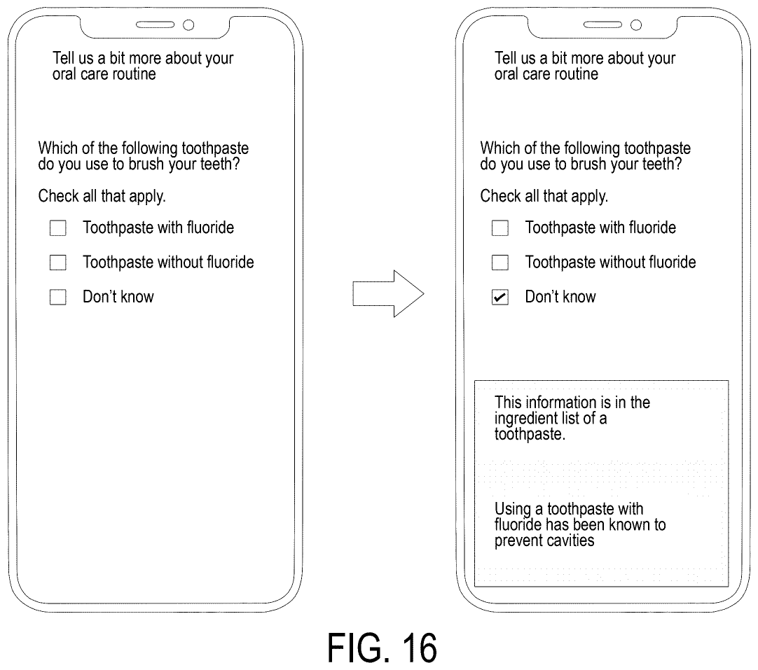

FIG. 16 illustrates an example diagram of an oral health assessment user interface in which a dental user subject replies to a survey question and receives a comment on the reply.

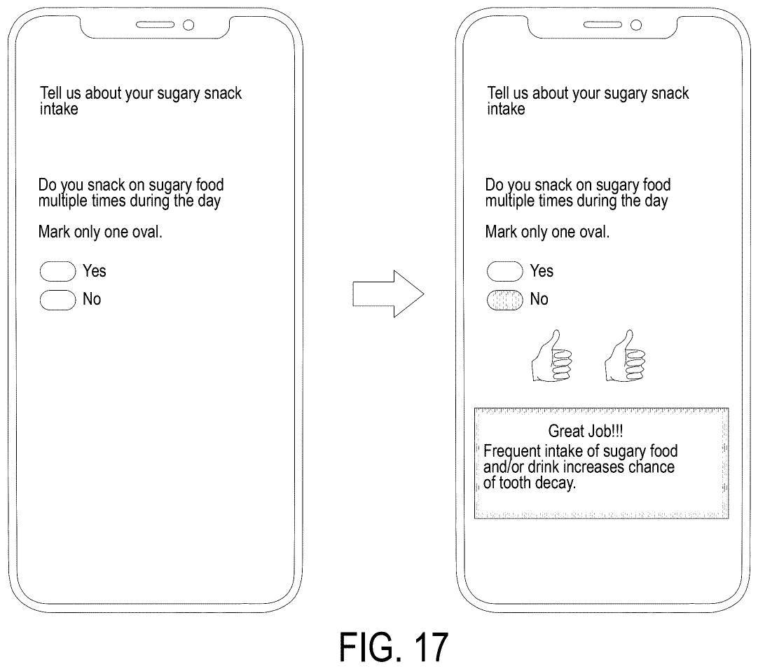

FIG. 17 illustrates an example diagram of an oral health assessment user interface in which a dental user subject replies to a survey question and receives a comment on the reply.

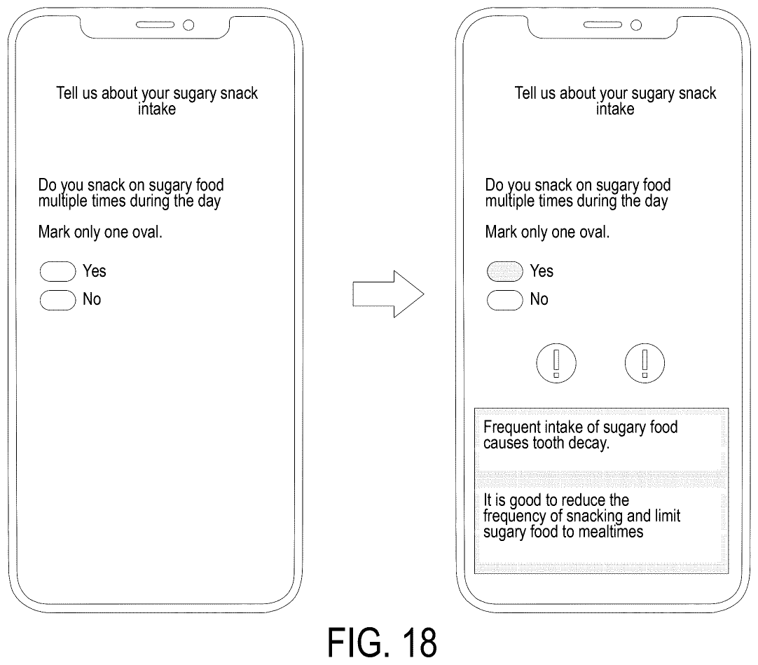

FIG. 18 illustrates an example diagram of an oral health assessment user interface in which a dental user subject replies to a survey question and receives a comment on the reply.

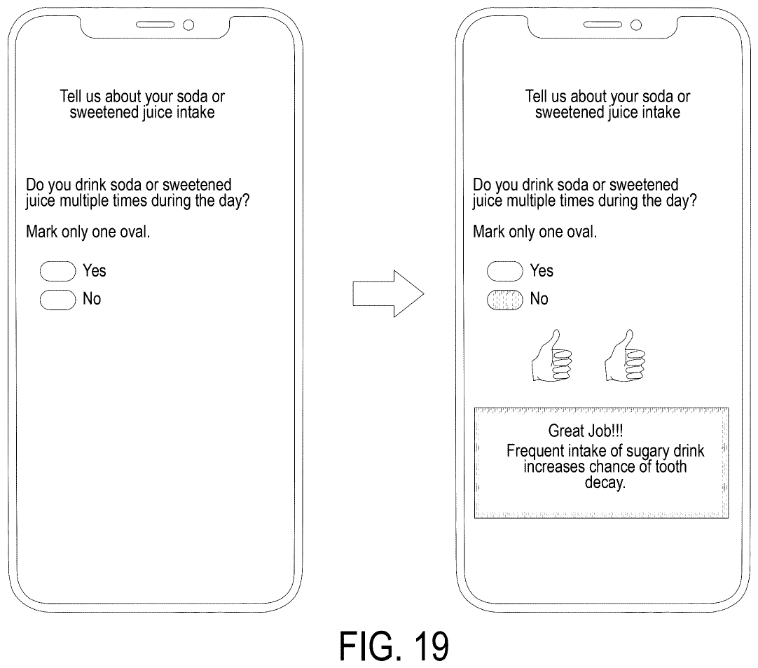

FIG. 19 illustrates an example diagram of an oral health assessment user interface in which a dental user subject replies to a survey question and receives a comment on the reply.

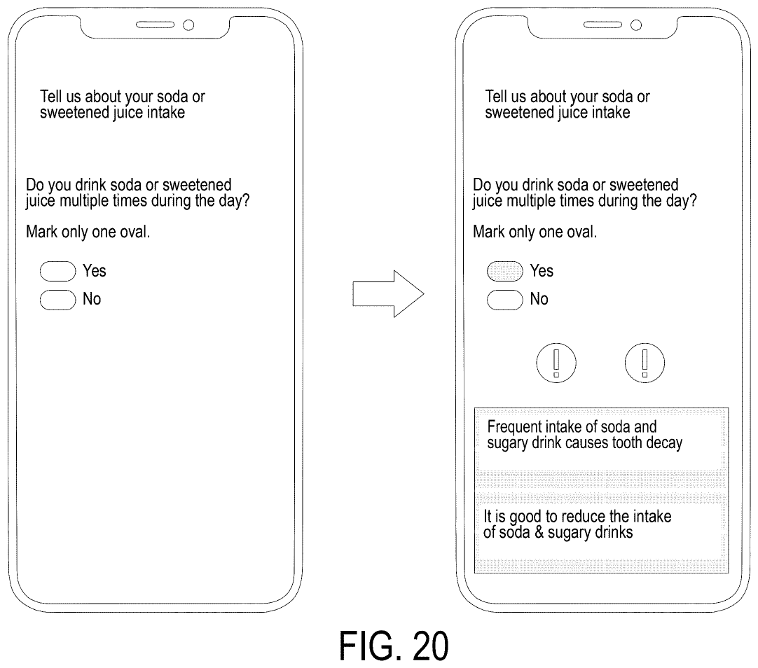

FIG. 20 illustrates an example diagram of an oral health assessment user interface in which a dental user subject replies to a survey question and receives a comment on the reply.

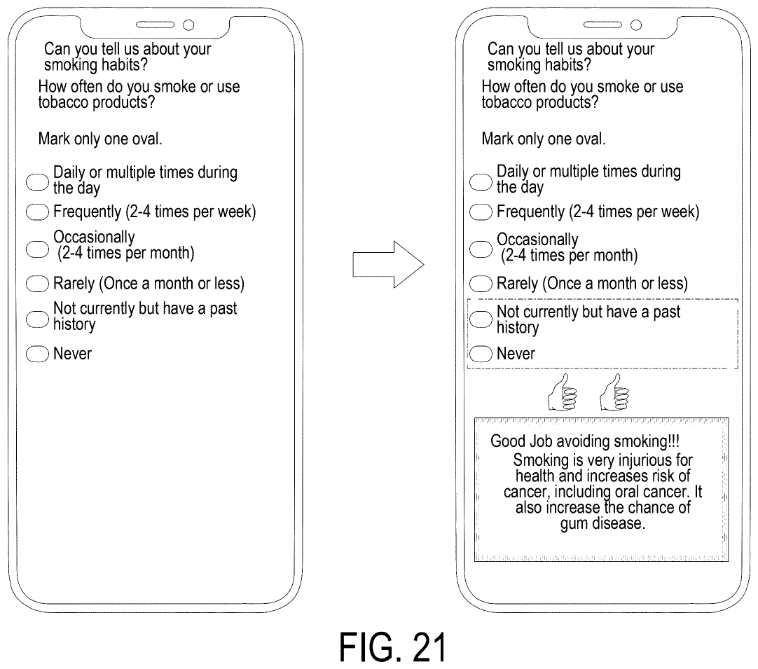

FIG. 21 illustrates an example diagram of an oral health assessment user interface illustrating a comment for a reply to two possible survey answers.

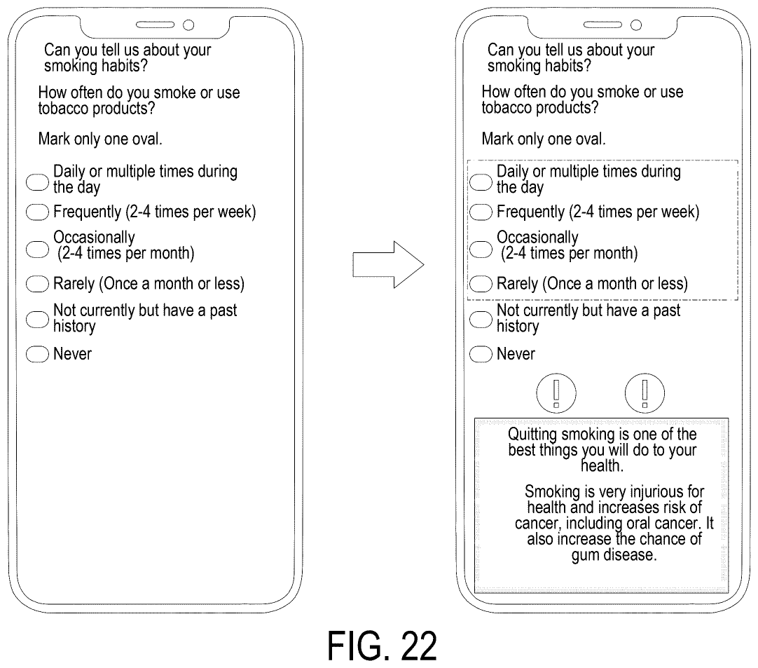

FIG. 22 illustrates an example diagram of an oral health assessment user interface illustrating a comment for a reply to four possible survey answers.

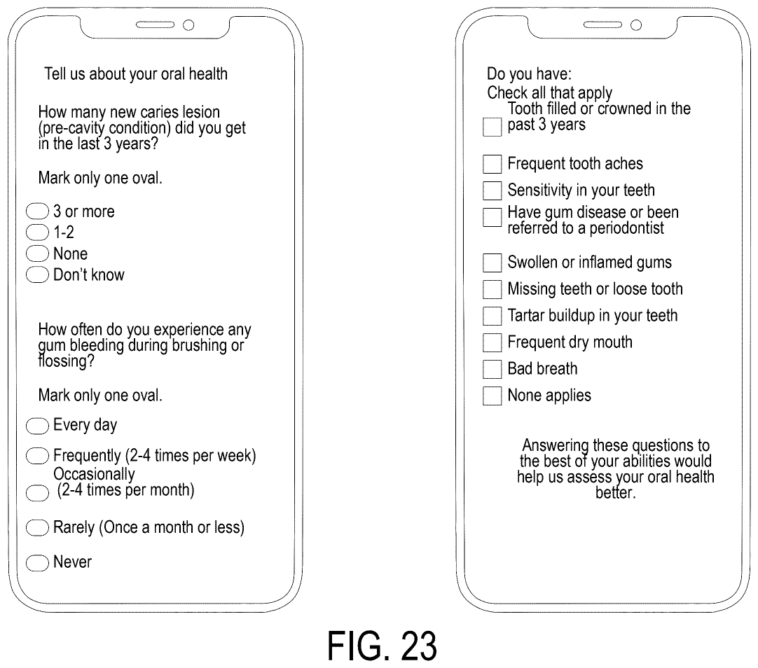

FIG. 23 illustrates an example diagram of an oral health assessment user interface in which a dental user subject is presented with at least three survey questions.

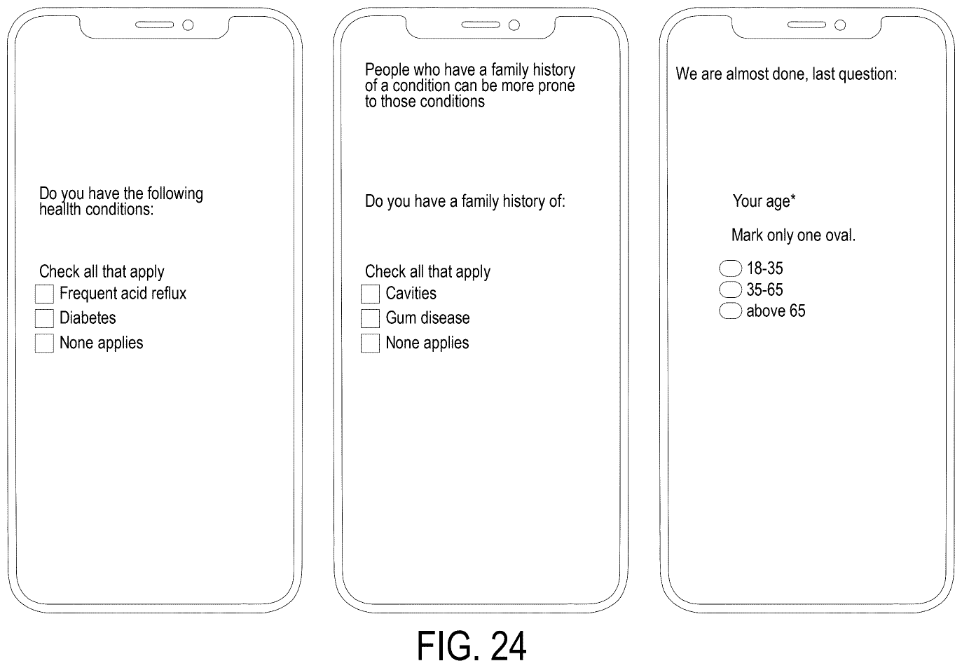

FIG. 24 illustrates an example diagram of an oral health assessment user interface in which a dental user subject is presented with at least three survey questions.

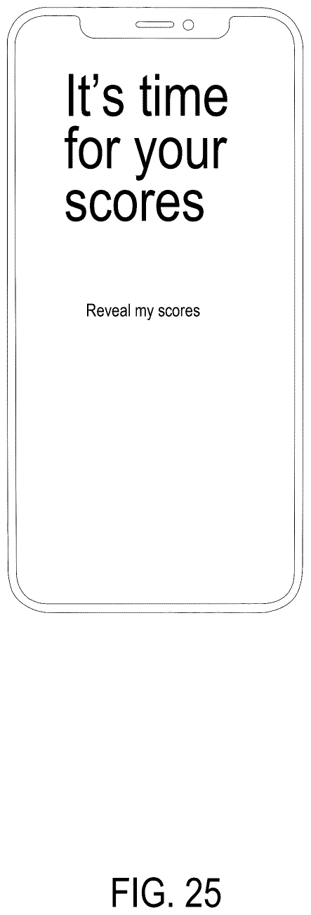

FIG. 25 illustrates an example diagram of an oral health assessment user interface in which a dental user subject is presented with a selection to produce one or more dental condition scores.

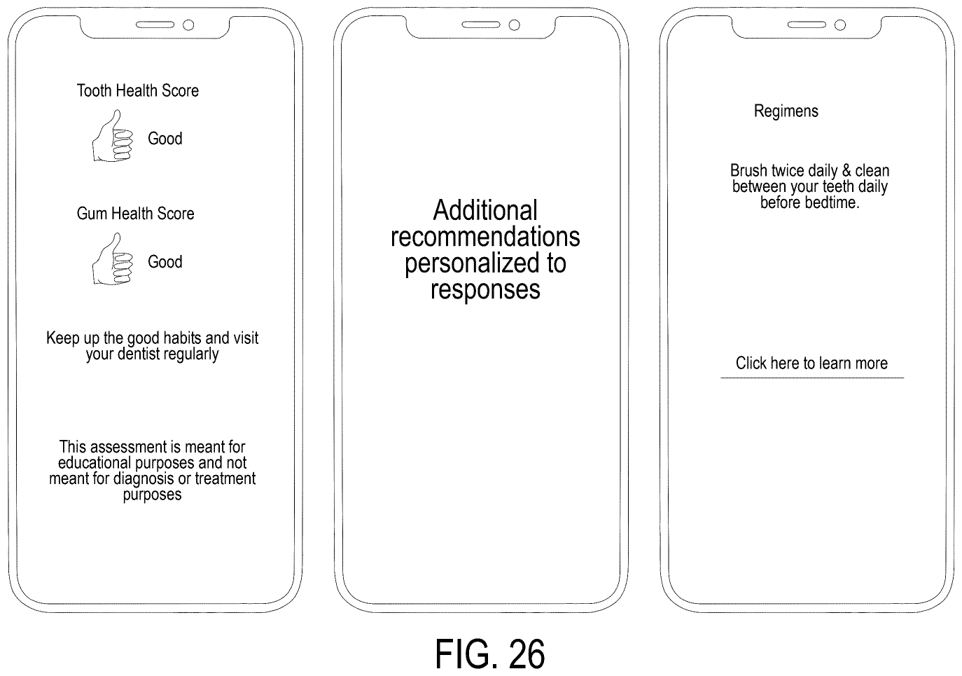

FIG. 26 illustrates an example diagram of an oral health assessment user interface in which a dental user subject is presented with two dental condition scores, one or more personalized recommendations, and links to information/literature regarding oral health.

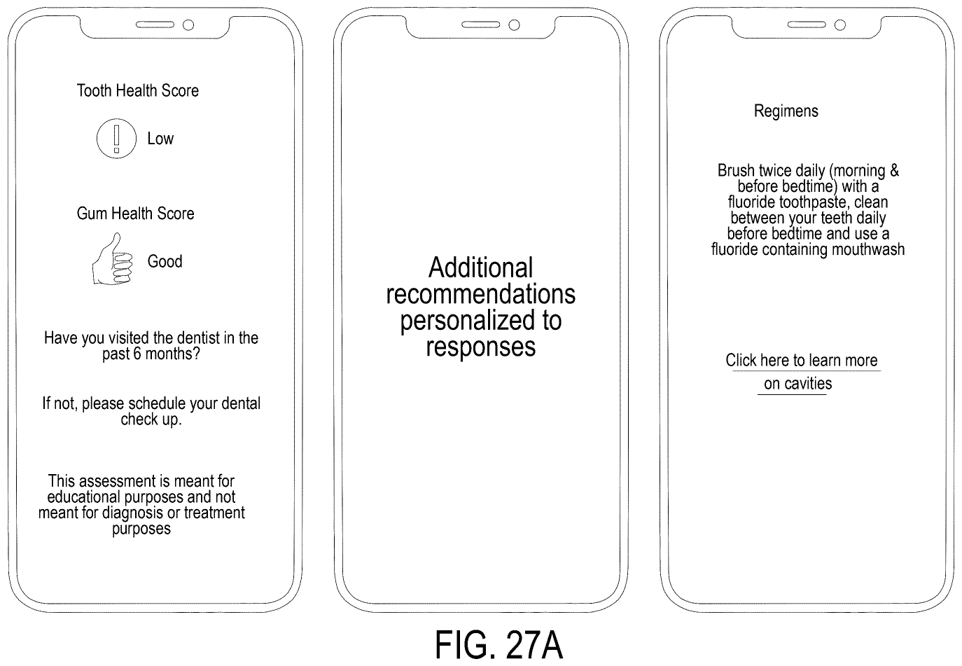

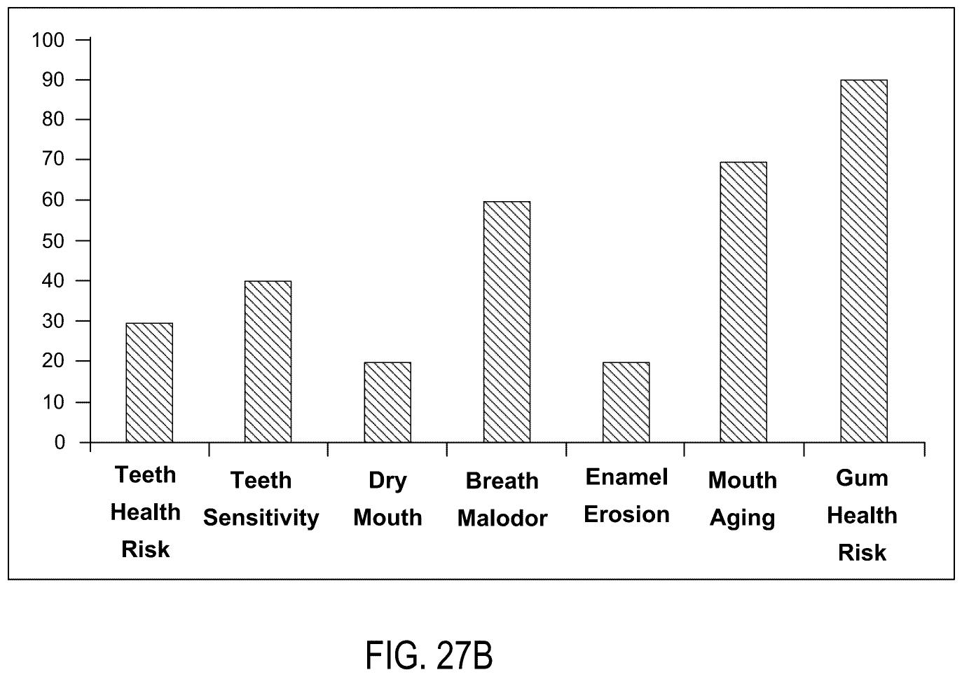

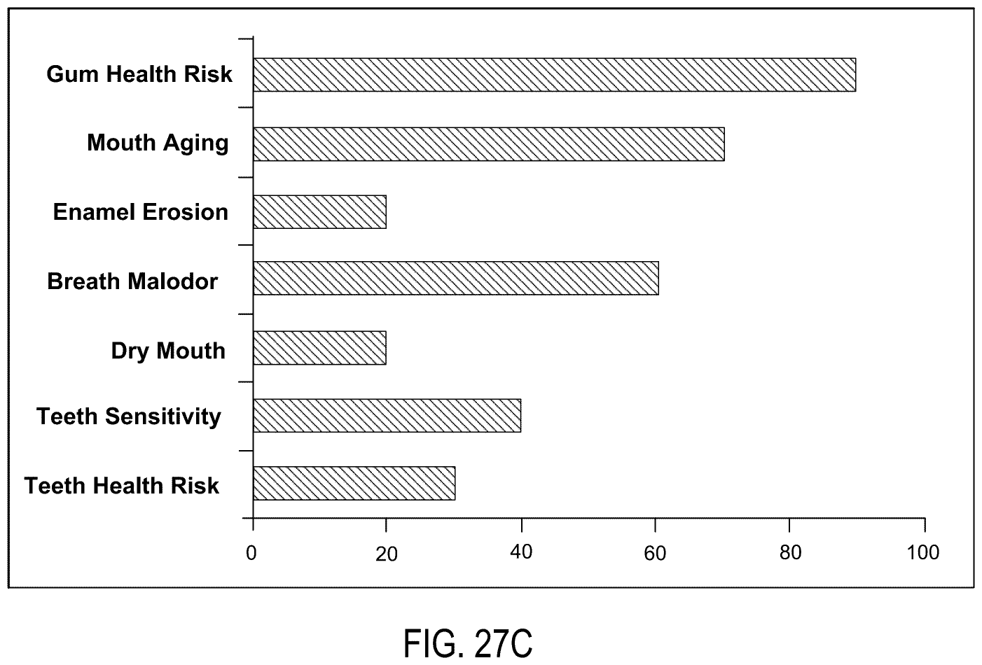

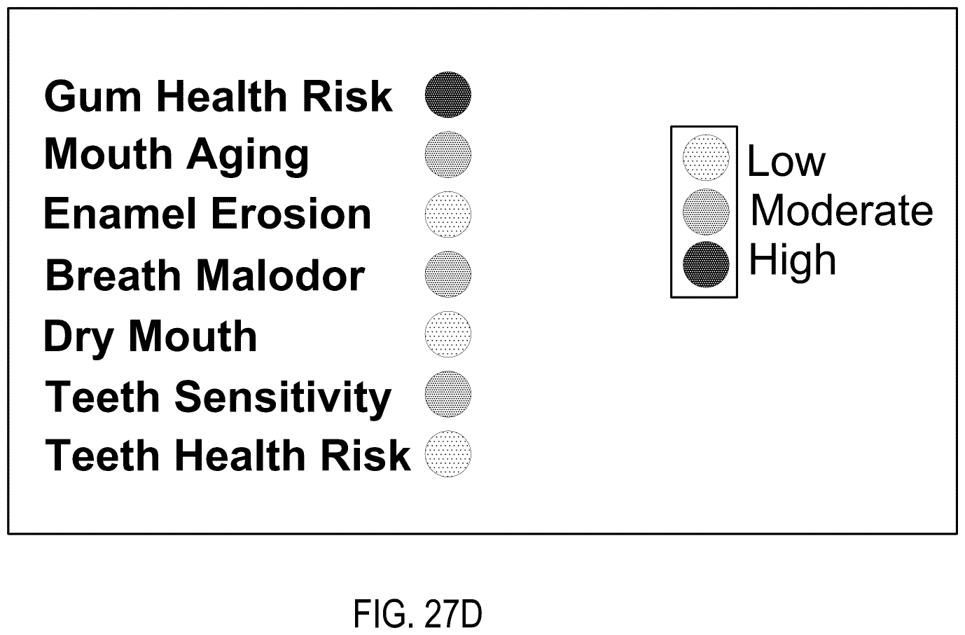

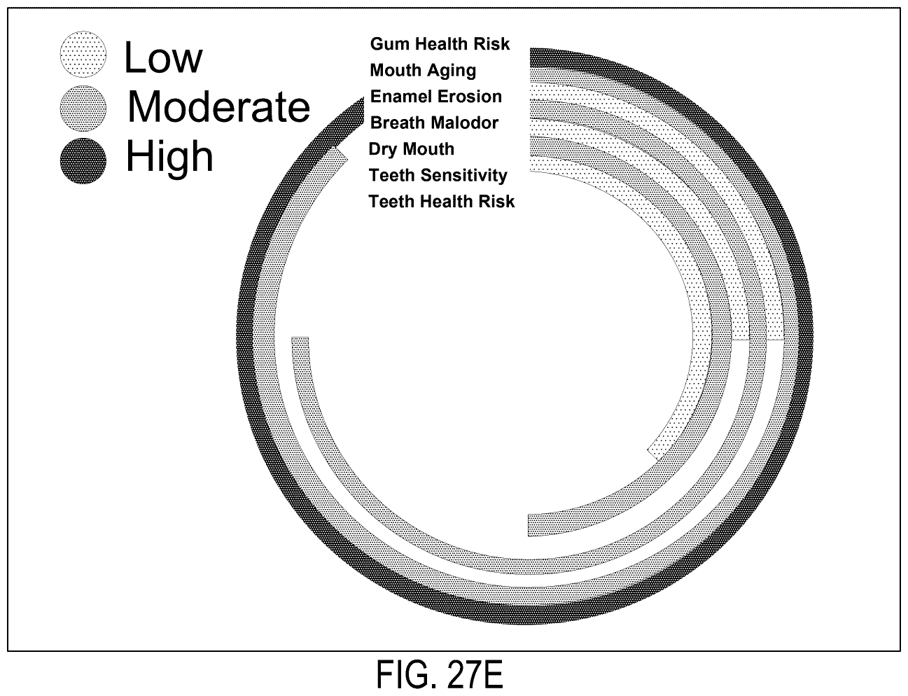

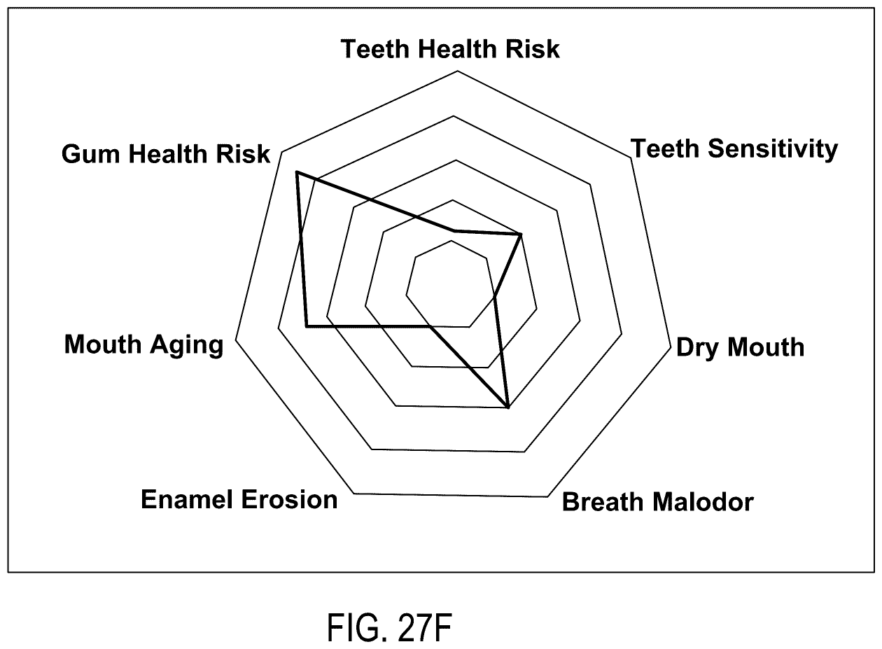

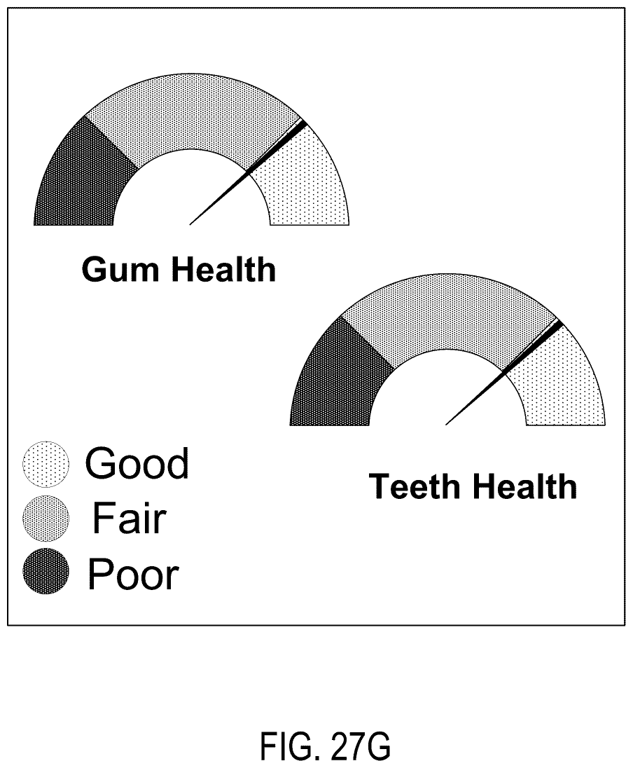

FIG. 27A illustrates an example diagram of an oral health assessment user interface in which a dental user subject is presented with two dental condition scores, one or more personalized recommendations, and links to information/literature regarding oral health.

FIG. 27B illustrates an example diagram of an oral health assessment that a dental user subject may be presented with that includes a vertical bar chart indicator with one or more dental condition/risk scores.

FIG. 27C illustrates an example diagram of an oral health assessment that a dental user subject may be presented with that includes a horizontal bar chart indicator with one or more dental condition/risk scores.

FIG. 27D illustrates an example diagram of an oral health assessment that a dental user subject may be presented with that includes a traffic light-type indicator with one or more dental condition/risk warning levels.

FIG. 27E illustrates an example diagram of an oral health assessment that a dental user subject may be presented with that includes a racetrack-type indicator with one or more dental condition/risk warning levels. A racetrack chart is a circular bar plot where each circle indicates a score/risk/condition for a given indication. Higher scores/risks/conditions, moderate scores/risks/conditions, and low scores/risks/conditions are differentiated by different patterns.

FIG. 27F illustrates an example diagram of an oral health assessment that a dental user subject may be presented with that includes a spider chart-type indicator with one or more dental condition/risk warning levels.

FIG. 27G illustrates an example diagram of an oral health assessment that a dental user subject may be presented with that includes one or more fuel gauge chart indicators with one or more dental condition/risk warning levels.

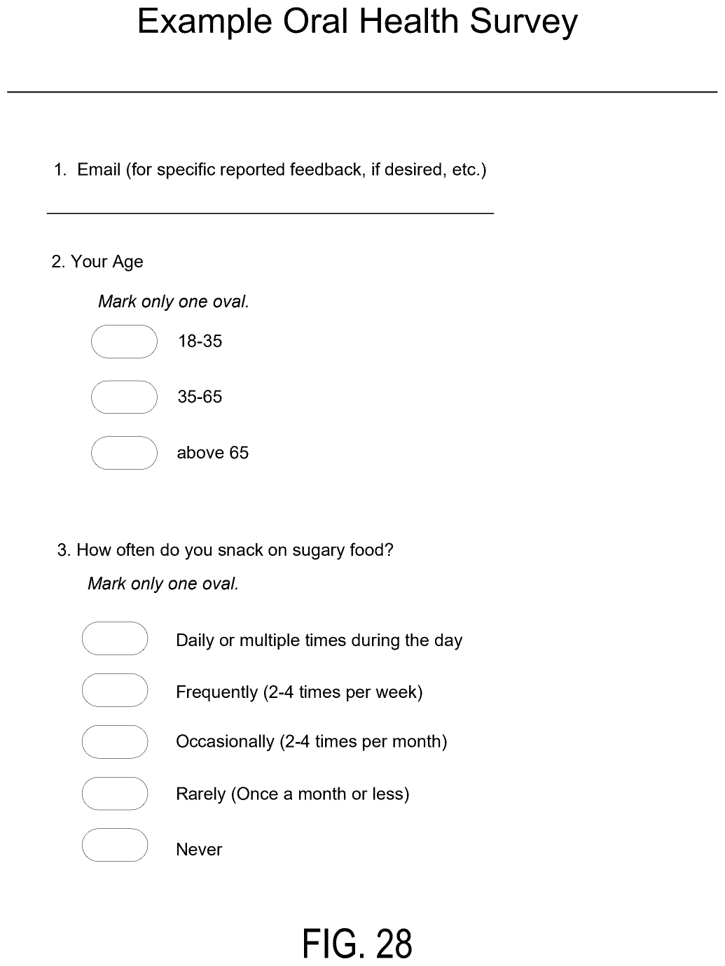

FIG. 28 illustrates three questions of an example oral health survey that may be presented to dental study subjects and/or dental user subjects. In one or more scenarios, the order and/or number of the questions may be changed and/or placed into different subsets. For example, questions may be put into different subsets, perhaps starting with less personal questions and then going into somewhat more personal questions. For example, in different subsets of questions, perhaps age may be placed first then moving toward oral care habits, then general habits, then dental history, and/or medical history, etc. Other potential orders of questions are contemplated. In one or more scenarios, different questions and/or different numbers of questions may be used for different dental patient users, wherein one or more questions may be, or might not be, weighted more (or less) in the one or more ML algorithm(s)/model(s) processing. The one or more ML algorithm(s)/model(s) may be trained with the one or more subsets of questions, which may be directed to one or more types of dental patient users, for example.

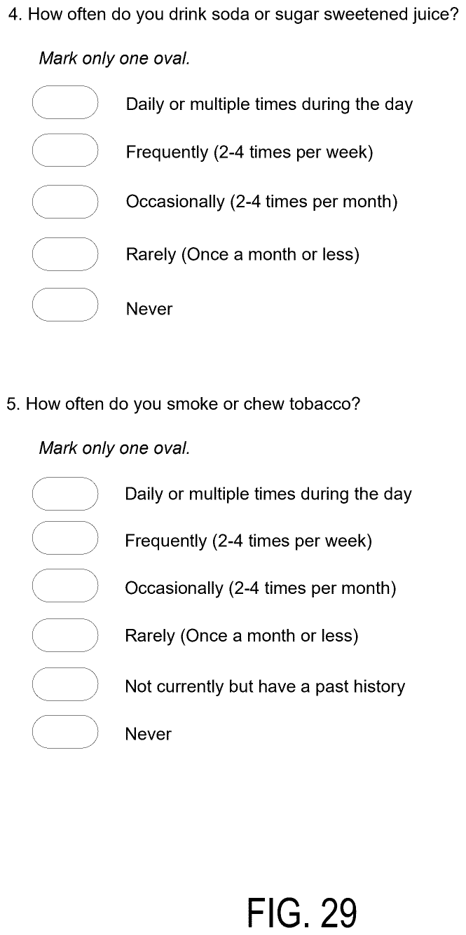

FIG. 29 illustrates two questions of an example oral health survey that may be presented to dental study subjects and/or dental user subjects.

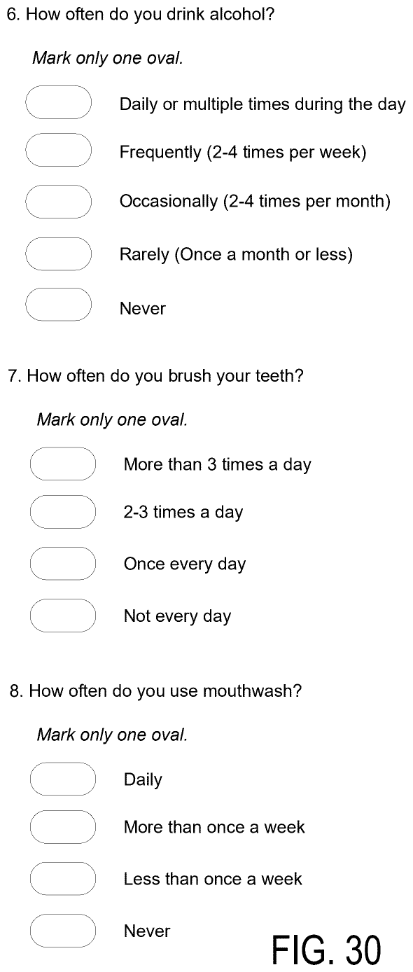

FIG. 30 illustrates three questions of an example oral health survey that may be presented to dental study subjects and/or dental user subjects.

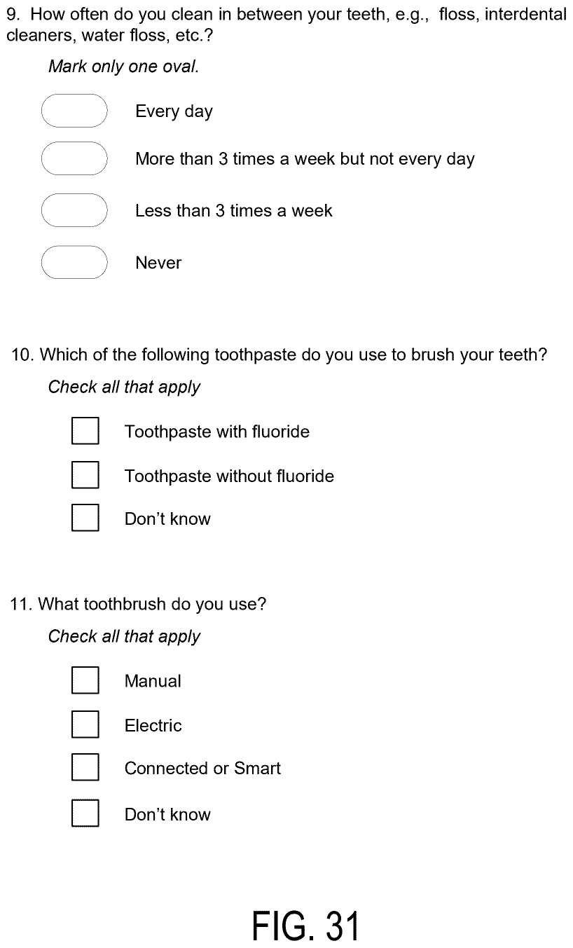

FIG. 31 illustrates three questions of an example oral health survey that may be presented to dental study subjects and/or dental user subjects.

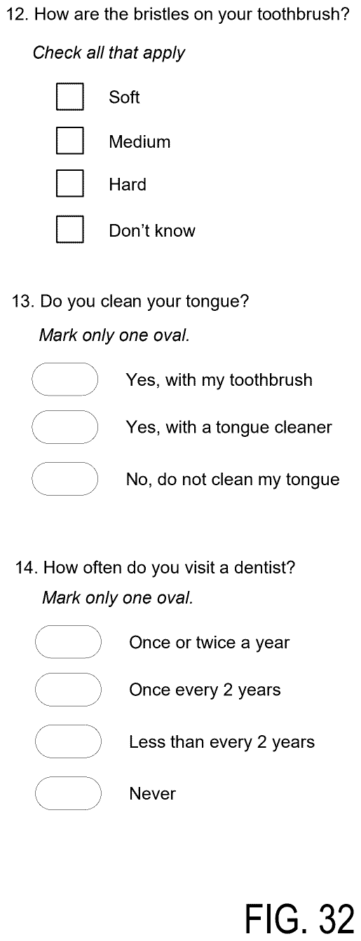

FIG. 32 illustrates three questions of an example oral health survey that may be presented to dental study subjects and/or dental user subjects.

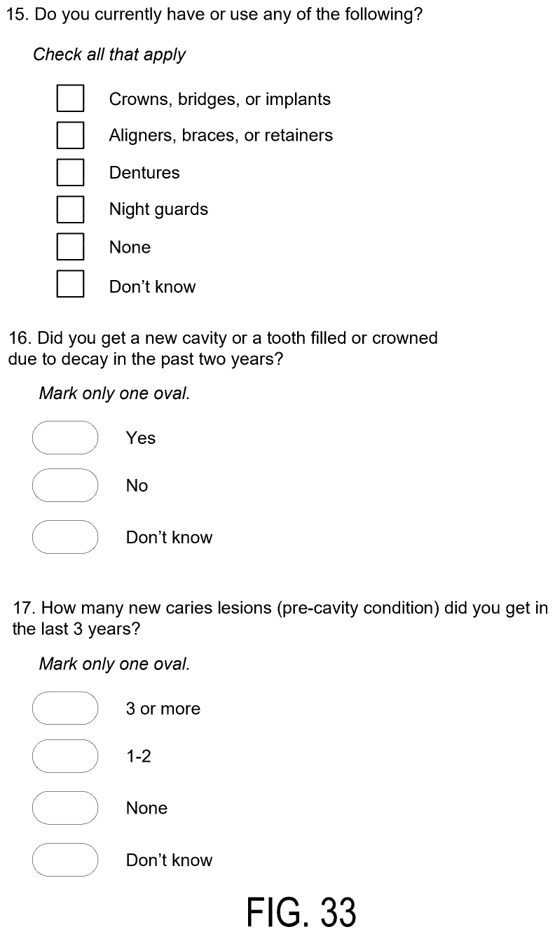

FIG. 33 illustrates three questions of an example oral health survey that may be presented to dental study subjects and/or dental user subjects.

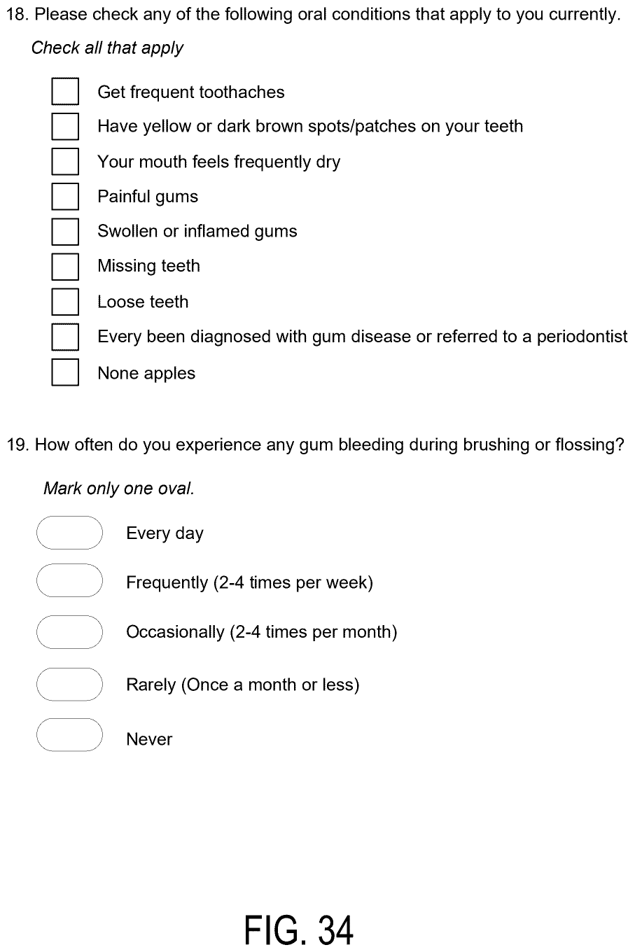

FIG. 34 illustrates two questions of an example oral health survey that may be presented to dental study subjects and/or dental user subjects.

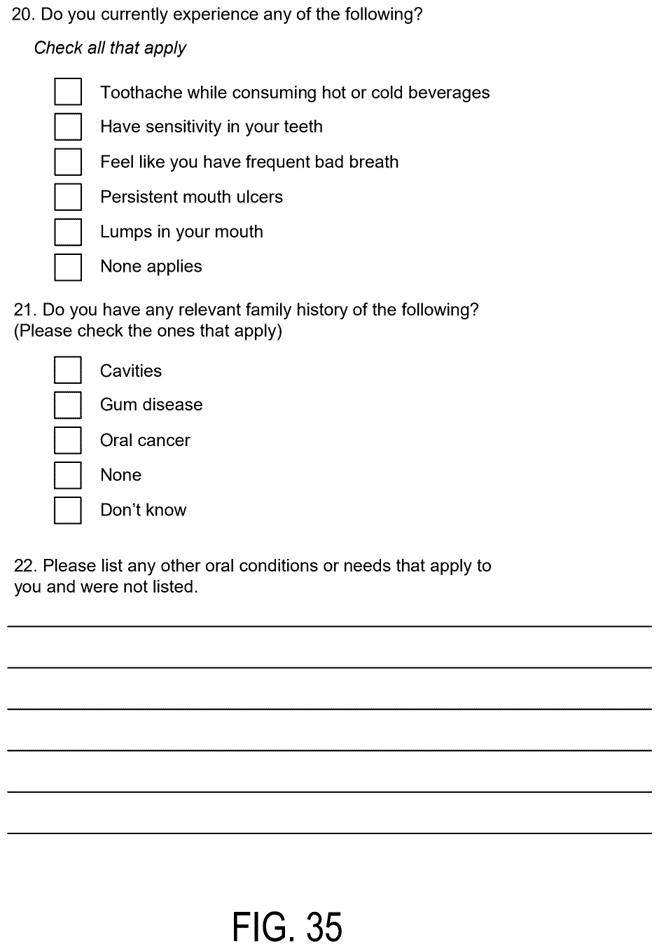

FIG. 35 illustrates three questions of an example oral health survey that may be presented to dental study subjects and/or dental user subjects.

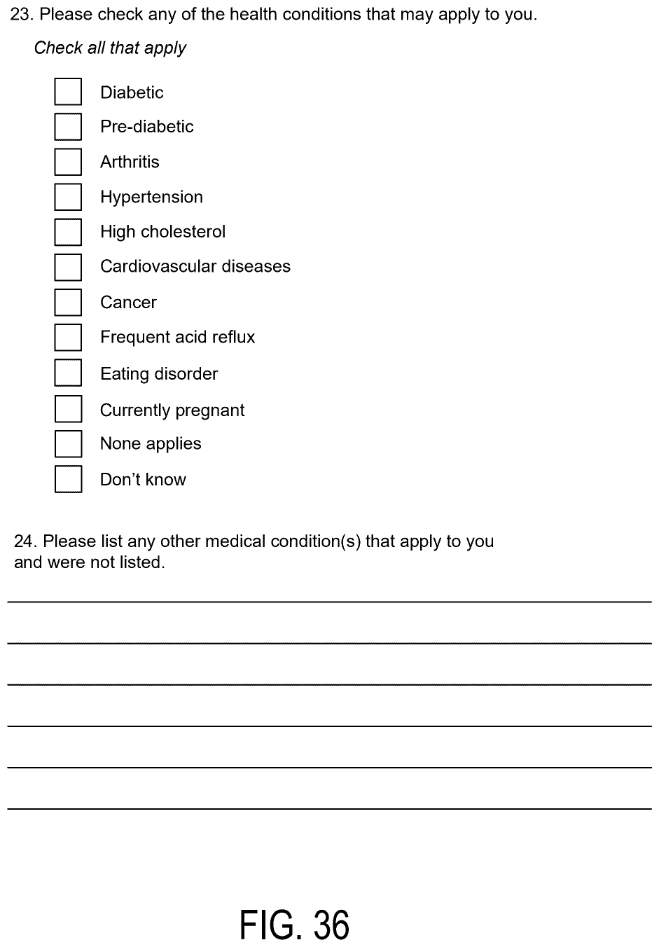

FIG. 36 illustrates two questions of an example oral health survey that may be presented to dental study subjects and/or dental user subjects.

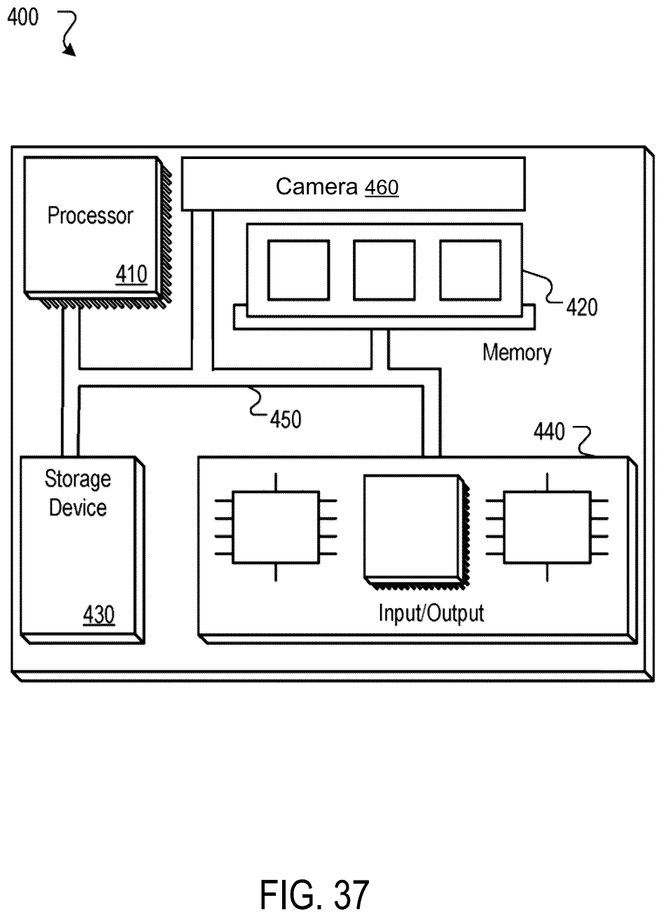

FIG. 37 is a block diagram of a hardware configuration of an example device that may function as a process control device/logic controller, such as a dental user subject device, a dental study subject device, and/or a machine-learning algorithm processing device, among other devices. The hardware configuration 400 may be operable to facilitate delivery of information from an internal server of a device. The hardware configuration 400 can include a processor 410, a memory 420, a storage device 430, and/or an input/output device 440. One or more of the components 410, 420, 430, and 440 can, for example, be interconnected using a system bus 450. The processor 410 can process instructions for execution within the hardware configuration 400. The processor 410 can be a single-threaded processor or the processor 410 can be a multi-threaded processor. The processor 410 can be capable of processing instructions stored in the memory 420 and/or on the storage device 430.

The memory 420 can store information within the hardware configuration 400. The memory 420 can be a computer-readable medium (CRM), for example, a non-transitory CRM. The memory 420 can be a volatile memory unit, and/or can be a non-volatile memory unit.

The storage device 430 can be capable of providing mass storage for the hardware configuration 400. The storage device 430 can be a computer-readable medium (CRM), for example, a non-transitory CRM. The storage device 430 can, for example, include a hard disk device, an optical disk device, flash memory and/or some other large capacity storage device. The storage device 430 can be a device external to the hardware configuration 400.

The input/output device 440 may provide input/output operations for the hardware configuration 400. The input/output device 440 (e.g., a transceiver device) can include one or more of a network interface device (e.g., an Ethernet card), a serial communication device (e.g., an RS-232 port), one or more universal serial bus (USB) interfaces (e.g., a USB 2.0 port) and/or a wireless interface device (e.g., an 802.11 card). The input/output device can include driver devices configured to send communications to, and/or receive communications from one or more networks (not shown). The input/output device 400 may be in communication with one or more input/output modules (not shown) that may be proximate to the hardware configuration 400 and/or may be remote from the hardware configuration 400. The one or more output modules may provide input/output functionality in the digital signal form, discrete signal form, TTL form, analog signal form, serial communication protocol, fieldbus protocol communication and/or other open or proprietary communication protocol, and/or the like.

The camera device 460 may provide digital video input/output capability for the hardware configuration 400. The camera device 460 may communicate with any of the elements of the hardware configuration 400, perhaps for example via system bus 450. The camera device 460 may capture digital images and/or may scan images/light of various kinds, such as Universal Product Code (UPC) codes and/or Quick Response (QR) codes, reflected light from a target area, e.g., oral cavity, legs, arm pits, etc., and/or excited fluorescence light from a target area, e.g., oral cavity, legs, arm pits, etc., for example, among other images/light as described herein. In one or more scenarios, the camera device 460 may be the same and/or substantially similar to any of the other camera devices as may be described herein.

The camera device 460 may include at least one microphone device and/or at least one speaker device (not shown). The input/output of the camera device 460 may include audio signals/packets/components, perhaps for example separate/separable from, or in some (e.g., separable) combination with, the video signals/packets/components the camera device 460.

The camera device 460 may be in wired and/or wireless communication with the hardware configuration 400. In one or more scenarios, the camera device 460 may be external to the hardware configuration 400. In one or more scenarios, the camera device 460 may be internal to the hardware configuration 400.

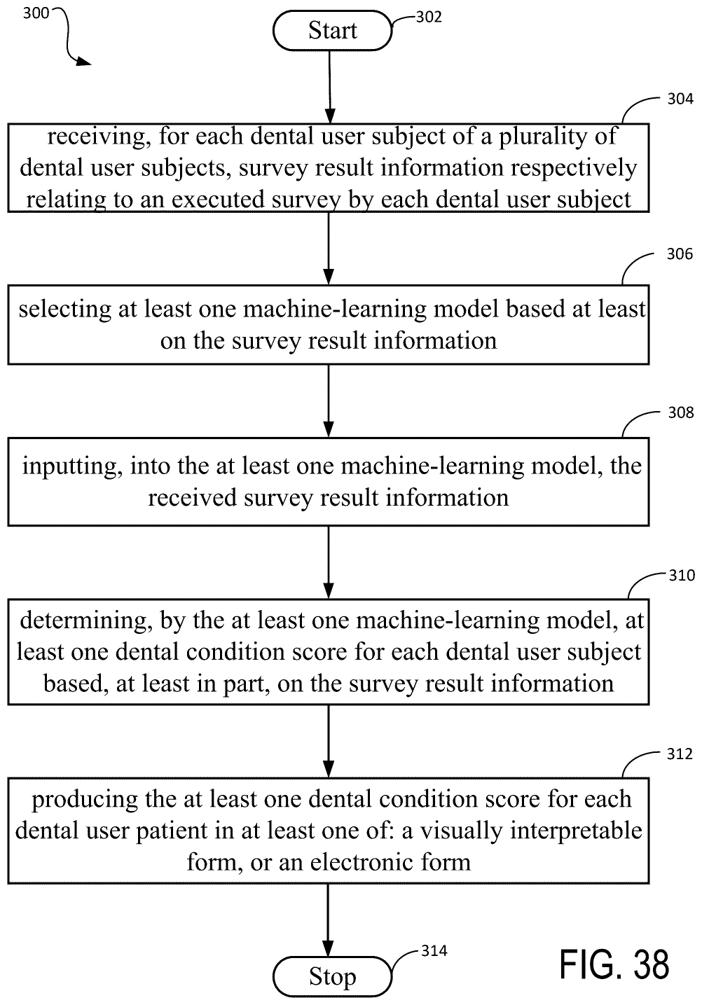

FIG. 38 is a flowchart of an example technique 300 for determining at least one dental condition score for a dental user patient based on an executed survey. At 302, one or more techniques may start. At 304, one or more techniques may comprise receiving, for each dental user subject of a plurality of dental user subjects, survey result information respectively relating to an executed survey by each dental user subject. At 306, one or more techniques may comprise selecting at least one machine-learning model based at least on the survey result information. At 308, one or more techniques may comprise inputting, into the at least one machine-learning model, the received survey result information. At 310, one or more techniques may comprise determining, by the at least one machine-learning model, at least one dental condition score for each dental user subject based, at least in part, on the survey result information. At 312, one or more techniques producing the at least one dental condition score for each dental user patient in at least one of a visually interpretable form, and/or an electronic form. At 314, one or more techniques may stop and/or restart.

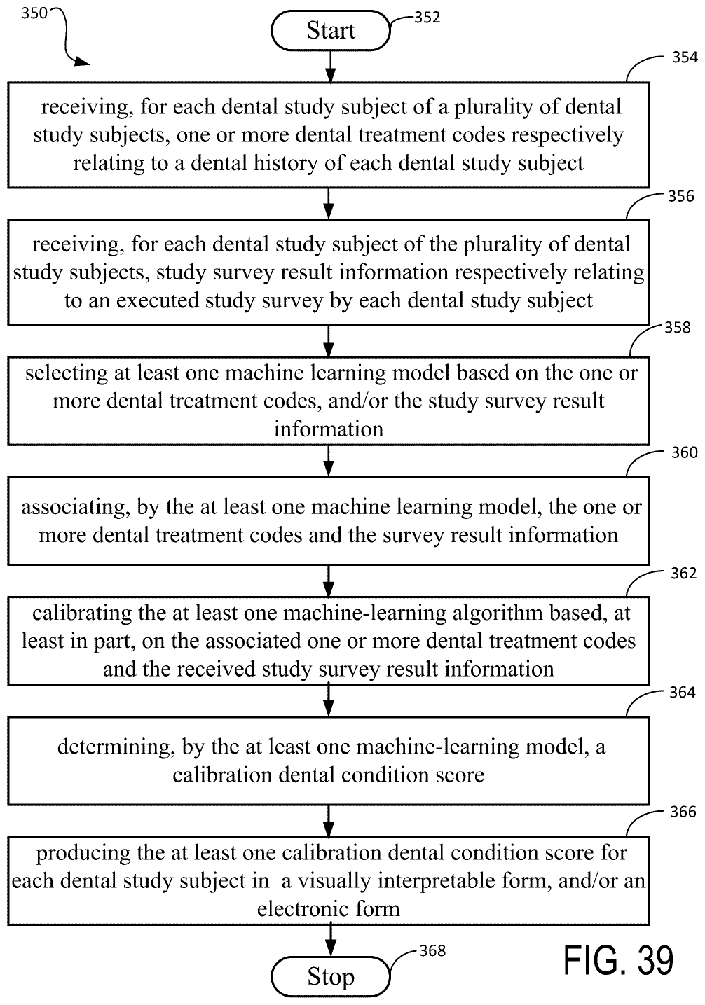

FIG. 39 is a flowchart of an example technique 350 for calibration of one or more machine-learning algorithms to produce a dental calibration score for a dental study subject. At 352, one or more techniques may start. At 354, one or more techniques may comprise receiving, for each dental study subject of a plurality of dental study subjects, one or more dental treatment codes respectively relating to a dental history of each dental study subject. At 356, one or more techniques may comprise receiving, for each dental study subject of the plurality of dental study subjects, study survey result information respectively relating to an executed study survey by each dental study subject. At 358, one or more techniques may comprise selecting at least one machine learning model based on the one or more dental treatment codes, and/or the study survey result information. At 360, one or more techniques may comprise associating, by the at least one machine learning model, the one or more dental treatment codes and the survey result information. At 362, one or more techniques may comprise calibrating the at least one machine-learning algorithm based, at least in part, on the associated one or more dental treatment codes and the received study survey result information. At 364, one or more techniques may comprise determining, by the at least one machine-learning model, a calibration dental condition score. At 366, one or more techniques may comprise producing the at least one calibration dental condition score for each dental study subject in a visually interpretable form, and/or an electronic form. At 368, one or more techniques may stop and/or restart.

In view of the descriptions provided herein, and in view of FIG. 1 to FIG. 39, the present disclosure describes one or more techniques for conducting oral health assessments, including one or more methods and/or devices therefore. In one or more scenarios, one or more computer-implemented methods may comprise receiving, for each dental user subject of a plurality of dental user subjects, survey result information respectively relating to an executed survey by each dental user subject. One or more methods may comprise selecting at least one machine-learning model based at least on the survey result information. One or more methods may comprise inputting, into the at least one machine-learning model, the received survey result information.

One or more methods may comprise determining, by the at least one machine-learning model, at least one dental condition score for each dental user subject based, at least in part, on the survey result information. One or more methods may comprise producing the at least one dental condition score for each dental user patient in a visually interpretable form, and/or an electronic form. One or more of the method elements may be performed by one or more processing devices. In one or more scenarios, the receiving the survey result information may comprise presenting, to each dental user subject, a survey comprising a plurality of questions via an electronic interface device. One or more methods may comprise presenting, to each dental user subject, a predetermined selection of answer choices for at least some of the plurality of questions. One or more methods may comprise receiving, via the electronic interface device, a dental user subject selected answer choice for the at least some of the plurality of questions.

In one or more scenarios, inputting the received survey result information may comprise converting the dental user subject selected answer choice for each of the at least some of the plurality of questions to a respective numerical value. One or more methods may comprise inputting, into the at least one machine-learning model, each numerical value respectively corresponding to the dental user subject selected answer choice for each of the at least some of the plurality of questions.

In one or more scenarios, one or more methods may comprise determining, by the one or more processing devices, at least one reply comment for each dental user subject selected answer choice for the at least some of the plurality of questions. One or more methods may comprise producing one or more of the at least one reply comments in a visually interpretable form, and/or an electronic form.

In one or more scenarios, the at least one reply comment for each dental user subject selected answer choice may be based on a predetermined correspondence between the at least one reply comment and the respective dental user subject selected answer choice.

In one or more scenarios, the at least one reply comment for each dental user subject selected answer choice may provide the dental user subject with constructive non-medical feedback corresponding to the dental user subject selected answer choice, and/or a non-medical affirmation corresponding to the dental user subject selected answer choice.

In one or more scenarios, the constructive non-medical feedback corresponding to the dental user subject selected answer choice, and/or the non-medical affirmation corresponding to the dental user subject selected answer choice may comprise a text message, an alpha-numeric message, and/or one or more symbols.

In one or more scenarios, selecting at least one machine-learning model may comprise selecting a first machine-learning model based at least on the survey result information. The first machine-learning model may correspond to gum disease analysis. One or more methods may comprise selecting a second machine-learning model based at least on the survey result information. The second machine-learning model may correspond to tooth/teeth condition analysis.

In one or more scenarios, determining the at least one dental condition score for each dental user subject may comprise determining, by the first machine-learning model, a first dental condition score corresponding to gum disease for each dental user subject based, at least in part, on the survey result information. One or more methods may comprise determining, by the second machine-learning model, a second dental condition score corresponding to tooth/teeth condition for each dental user subject based, at least in part, on the survey result information.

In one or more scenarios, the first dental condition score may be at least one of a low or bad gum health score, a medium or average gum health score, or a high or good gum health score. The second dental condition score may be at least one of a low or bad teeth health score, a medium or average teeth health score, or a high or good tooth/teeth health score. Other kinds of scores, such as risk scores and/or heath scores, are contemplated such as binary, low, moderate, high, scales of 1 to N (e.g., N being an integer or real number), for example, among other kinds of scores.

In one or more scenarios, one or more methods may comprise determining, by the at least one machine-learning model, one or more parameters having a relatively high relevance to the at least one dental condition score for each dental user patient based, at least in part, on the survey result information. One or more methods may comprise producing at least some of the one or more parameters for the at least one dental condition score for each dental user patient in a visually interpretable form, and/or an electronic form.

In one or more scenarios, one or more methods may comprise determining, by the one or more processing devices, at least one score comment corresponding to the at least one dental condition score for each dental user patient. The at least one score comment may comprise constructive non-medical feedback corresponding to the at least one dental condition score, and/or a non-medical affirmation corresponding to the at least one dental condition score. One or more methods may comprise producing the at least one score comment in a visually interpretable form, and/or an electronic form. The constructive non-medical feedback corresponding to the at least one dental condition score, and/or the non-medical affirmation corresponding to the at least one dental condition score, may comprise a text message, an alpha-numeric message, and/or one or more symbols.

In one or more scenarios, the at least one score comment may comprise one or more of a lifestyle suggestion, one or more learning materials, one or more dental care products, and/or one or more coaching suggestions.

In one or more scenarios, selecting at least one machine-learning model may comprise selecting a third machine-learning model based at least on the survey result information. The third machine-learning model may correspond to breath odor analysis. One or more methods may comprise selecting a fourth machine-learning model based at least on the survey result information. The fourth machine-learning model may correspond to one or more of dentition/gum sensitivity analysis, dry mouth analysis, enamel erosion analysis, and/or mouth aging analysis.

In one or more scenarios, one or more methods may comprise receiving, for each dental study subject of a plurality of dental study subjects, one or more dental treatment codes respectively relating to a dental history of each dental study subject. One or more methods may comprise receiving, for each dental study subject of the plurality of dental study subjects, study survey result information respectively relating to an executed study survey by each dental study subject. One or more methods may comprise selecting at least one machine learning model based on the one or more dental treatment codes, and/or the study survey result information.

One or more methods may comprise associating, by the at least one machine learning model, the one or more dental treatment codes and/or the survey result information. One or more methods may comprise calibrating the at least one machine-learning algorithm based, at least in part, on the associated one or more dental treatment codes and/or the received study survey result information. One or more methods may comprise determining, by the at least one machine-learning model, a calibration dental condition score. One or more methods may comprise producing the at least one calibration dental condition score for each dental study subject in a visually interpretable form, and/or an electronic form. One or more of the method elements may be performed by one or more processing devices.

In one or more scenarios, calibrating the at least one machine-learning model may comprise iteratively updating one or more parameters of the at least one machine-learning algorithm to minimize an objective function, and/or a loss function, of the at least one machine-learning algorithm. One or more methods may comprise adjusting the one or more parameters of the at least one machine-learning model to reduce a deviation between the calibration dental condition score and the one or more dental treatment codes.

In one or more scenarios, associating the one or more dental treatment codes and the survey result information may comprise associating the one or more dental treatment codes respectively relating to a dental history of a respective dental study subject with the study survey result information relating to the executed study survey by the same respective dental study subject.

In one or more scenarios, selecting the at least one machine learning model may comprise determining a correspondence between the one or more dental treatment codes and a first machine-learning algorithm model corresponding to gum disease analysis, and/or a second machine-learning model corresponding to teeth condition analysis. One or more methods may comprise selecting the first machine-learning model, and/or the second machine-learning model, based on the determined correspondence.

In one or more scenarios, selecting the at least one machine-learning model may comprise determining a correspondence between the one or more dental treatment codes and a third machine-learning algorithm model corresponding to breath odor analysis, and/or a fourth machine-learning model corresponding to at least one of: dentition/gum sensitivity analysis, enamel erosion analysis, dry mouth analysis, and/or mouth aging analysis. One or more methods may comprise selecting the third machine-learning model, and/or the fourth machine-learning model, based on the determined correspondence. In one or more scenarios, there may be five or more machine-learning models, for example.

In one or more scenarios, the at least one machine-learning algorithm may be adjustable into an operational mode, or a learning mode. One or more methods may comprise adjusting the at least one machine-learning model into the learning mode and/or the operational mode.

In one or more scenarios, one or more techniques described herein may include computer-implemented methods and/or devices performing same. One or more methods may comprise receiving, for each dental study subject of the plurality of dental study subjects, study survey result information respectively relating to an executed study survey by each dental study subject. One or more methods may comprise receiving, for one or more dental study subject of the plurality of dental study subjects, one or more dental records comprising dental records for treatment of dental caries and/or periodontitis. One or more methods may comprise selecting at least one machine learning model based on the study survey result information, and/or the one or more dental records. One or more methods may comprise associating, by the at least one machine learning model, the one or more dental records and the survey result information. One or methods may comprise calibrating the at least one machine-learning algorithm based, at least in part, on the associated one or more dental records and the received study survey result information. One or methods may comprise determining, by the at least one machine-learning model, a calibration dental condition score. One or more methods may comprise producing the at least one calibration dental condition score for each dental study subject in a visually interpretable form, and/or an electronic form. One or more of the method elements may be performed by one or more processing devices.

In one or more scenarios, calibrating the at least one machine-learning model may comprise iteratively updating one or more parameters of the at least one machine-learning algorithm to minimize an objective function, and/or a loss function, of the at least one machine-learning algorithm, or adjusting the one or more parameters of the at least one machine-learning model to reduce a deviation between the calibration dental condition score and the one or more dental records.

In one or more scenarios, associating the one or more dental records and the survey result information may comprise associating the one or more dental records of the one or more respective dental study subjects with the study survey result information relating to the executed study survey by the same respective one or more dental study subjects.

In one or more scenarios, selecting the at least one machine learning model may comprise determining a correspondence between the one or more dental records and a first machine-learning algorithm model corresponding to gum disease analysis, and/or a second machine-learning model corresponding to teeth condition analysis. In one or more scenarios, selecting the at least one machine learning model may comprise selecting the first machine-learning model, and/or the second machine-learning model, based on the determined correspondence.

In one or more scenarios, selecting the at least one machine learning model may comprise determining a correspondence between the one or more dental records and a third machine-learning algorithm model corresponding to breath odor analysis, and/or a fourth machine-learning model corresponding to dentition/gum sensitivity analysis, enamel erosion analysis, dry mouth analysis, and/or mouth aging analysis. In one or more scenarios, selecting the at least one machine learning model may comprise selecting the third machine-learning model, and/or the fourth machine-learning model, based on the determined correspondence.

In one or more scenarios, the at least one machine-learning algorithm may be adjustable into an operational mode, and/or a learning mode. In one or more scenarios, one or more techniques may comprise adjusting the at least one machine-learning model into the learning mode.

In one or more scenarios, the one or more dental records may correspond to one or more dental study subjects that indicated on their respective executed study surveys an experience of gum disease, a teeth condition, breath odor, dentition/gum sensitivity, enamel erosion, dry mouth analysis, and/or mouth aging.

In one or more scenarios, the one or more dental records may correspond to one or more of at least three years of dental care history corresponding to the one or more dental study subjects that indicated on their respective executed study surveys an experience of at least one of: gum disease, teeth condition, breath odor, dentition/gum sensitivity, enamel erosion, dry mouth analysis, and/or mouth aging.

While the inventions have been described with respect to specific examples including presently preferred modes of carrying out the inventions, those skilled in the art will appreciate that there are numerous variations and permutations of the herein described systems and techniques. It is to be understood that other embodiments may be utilized and structural and functional modifications may be made without departing from the scope of the present inventions. Thus, the spirit and scope of the inventions should be construed broadly as set forth in the appended claims.

The subject matter of this disclosure, and components thereof, can be realized by instructions that upon execution cause one or more processing devices to carry out the processes and/or functions described herein. Such instructions can, for example, comprise interpreted instructions, such as script instructions, e.g., JavaScript or ECMAScript instructions, or executable code, and/or other instructions stored in a computer readable medium. C++, C#, and/or C, Python scripts and/or Zephyr RTOS may be used.

Implementations of the subject matter and/or the functional operations described in this specification and/or the accompanying figures can be provided in digital electronic circuitry, in computer software, firmware, and/or hardware, including the structures disclosed in this specification and their structural equivalents, and/or in combinations of one or more of them. The subject matter described in this specification can be implemented as one or more computer program products, e.g., one or more modules of computer program instructions encoded on a tangible program carrier for execution by, and/or to control the operation of, data processing apparatus.

A computer program (also known as a program, software, software application, script, or code) can be written in any form of programming language, including compiled or interpreted languages, and/or declarative or procedural languages. It can be deployed in any form, including as a stand-alone program or as a module, component, subroutine, and/or other unit suitable for use in a computing environment. A computer program may or might not correspond to a file in a file system. A program can be stored in a portion of a file that holds other programs and/or data (e.g., one or more scripts stored in a markup language document), in a single file dedicated to the program in question, and/or in multiple coordinated files (e.g., files that store one or more modules, sub programs, or portions of code). A computer program can be deployed to be executed on one computer or on multiple computers that may be located at one site or distributed across multiple sites and/or interconnected by a communication network.

The processes and/or logic flows described in this specification and/or in the accompanying figures may be performed by one or more programmable processors executing one or more computer programs to perform functions by operating on input data and/or generating output, thereby tying the process to a particular machine (e.g., a machine programmed to perform the processes described herein). The processes and/or logic flows can also be performed by, and apparatus can also be implemented as, special purpose logic circuitry, e.g., an FPGA (field programmable gate array) and/or an ASIC (application specific integrated circuit).

Computer readable media suitable for storing computer program instructions and/or data may include all forms of non-volatile memory, media and memory devices, including by way of example semiconductor memory devices (e.g., EPROM, EEPROM, and/or flash memory devices); magnetic disks (e.g., internal hard disks or removable disks); magneto optical disks; and/or CD ROM and DVD ROM disks. The processor and/or the memory can be supplemented by, or incorporated in, special purpose logic circuitry.

While this specification and the accompanying figures contain many specific implementation details, these should not be construed as limitations on the scope of any invention and/or of what may be claimed, but rather as descriptions of features that may be specific to described example implementations. Certain features that are described in this specification in the context of separate implementations can also be implemented in combination in perhaps one implementation. Various features that are described in the context of perhaps one implementation can also be implemented in multiple combinations separately or in any suitable sub-combination. Although features may be described above as acting in certain combinations and/or perhaps even (e.g., initially) claimed as such, one or more features from a claimed combination can in some cases be excised from the combination. The claimed combination may be directed to a sub-combination and/or variation of a sub-combination.

While operations may be depicted in the drawings in an order, this should not be understood as requiring that such operations be performed in the particular order shown and/or in sequential order, and/or that all illustrated operations be performed, to achieve useful outcomes. The described program components and/or systems can generally be integrated together in a single software product and/or packaged into multiple software products.

Examples of the subject matter described in this specification have been described. The actions recited in the claims can be performed in a different order and still achieve useful outcomes, unless expressly noted otherwise. For example, the processes depicted in the accompanying figures do not require the particular order shown, and/or sequential order, to achieve useful outcomes. Multitasking and parallel processing may be advantageous in one or more scenarios.

While the present disclosure has been illustrated and described in detail in the drawings and foregoing description, the same is to be considered as illustrative and not restrictive in character, it being understood that only certain examples have been shown and described, and that all changes and modifications that come within the spirit of the present disclosure are desired to be protected.

What is claimed is:

1. A computer-implemented method comprising:
(a) receiving, for each dental user subject of a plurality of dental user subjects, survey result information respectively relating to an executed survey by each dental user subject;
(b) selecting at least one machine-learning model based at least on the survey result information;
(c) inputting, into the at least one machine-learning model, the received survey result information;
(d) determining, by the at least one machine-learning model, at least one dental condition score for each dental user subject based, at least in part, on the survey result information; and
(e) producing the at least one dental condition score for each dental user patient in at least one of: a visually interpretable form, or an electronic form,
wherein at least steps (a)-(e) are performed by one or more processing devices.

2. The method of claim 1, wherein the receiving the survey result information further comprises:
(a-1) presenting, to each dental user subject, a survey comprising a plurality of questions via an electronic interface device;
(a-2) presenting, to each dental user subject, a predetermined selection of answer choices for at least some of the plurality of questions; and
(a-3) receiving, via the electronic interface device, a dental user subject selected answer choice for the at least some of the plurality of questions.

3. The method of claim 2, wherein the inputting the received survey result information further comprises:
(c-1) converting the dental user subject selected answer choice for each of the at least some of the plurality of questions to a respective numerical value; and
(c-2) inputting, into the at least one machine-learning model, each numerical value respectively corresponding to the dental user subject selected answer choice for each of the at least some of the plurality of questions.

4. The method of claim 3, further comprising:
(c-3) determining, by the one or more processing devices, at least one reply comment for each dental user subject selected answer choice for the at least some of the plurality of questions; and
(c-4) producing one or more of the at least one reply comments in at least one of: a visually interpretable form, or an electronic form.

5. The method of claim 4, wherein the at least one reply comment for each dental user subject selected answer choice is based on a predetermined correspondence between the at least one reply comment and the respective dental user subject selected answer choice.

6. The method of claim 4, wherein the at least one reply comment for each dental user subject selected answer choice provides the dental user subject with at least one of: constructive non-medical feedback corresponding to the dental user subject selected answer choice, or a non-medical affirmation corresponding to the dental user subject selected answer choice, wherein at least one of: the constructive non-medical feedback corresponding to the dental user subject selected answer choice, or the non-medical affirmation corresponding to the dental user subject selected answer choice comprises at least one of: a text message, an alpha-numeric message, or one or more symbols.

7. The method of claim 1, wherein the selecting at least one machine-learning model further comprises:
(b-1) selecting a first machine-learning model based at least on the survey result information, the first machine-learning model corresponding to gum disease analysis; and
(b-2) selecting a second machine-learning model based at least on the survey result information, the second machine-learning model corresponding to teeth condition analysis.

8. The method of claim 7, wherein the determining the at least one dental condition score for each dental user subject further comprises:
(d-1) determining, by the first machine-learning model, a first dental condition score corresponding to gum disease for each dental user subject based, at least in part, on the survey result information; and
(d-2) determining, by the second machine-learning model, a second dental condition score corresponding to teeth condition for each dental user subject based, at least in part, on the survey result information.

9. The method of claim 8, wherein at least one of:
the first dental condition score is at least one of: a low or bad gum health score, a medium or average gum health score, or a high or good gum health score, and the second dental condition score is at least one of: a low or bad teeth health score, a medium or average teeth health score, or a high or good teeth health score;
the first dental condition score is at least one of: a binary gum health score, a numerical gum health score, and/or a relative gum health score, and the second dental condition score is at least one of: a binary teeth health score, a numerical teeth health score, and/or a relative teeth health score; or
the first dental condition score is at least one of: a binary gum risk score, a numerical gum risk score, and/or a relative gum risk score, and the second dental condition score is at least one of: a binary teeth risk score, a numerical teeth risk score, and/or a relative teeth risk score.

10. The method of claim 1, further comprising:
(f) determining, by the at least one machine-learning model, one or more parameters having a relatively high relevance to the at least one dental condition score for each dental user patient based, at least in part, on the survey result information; and
(g) producing at least some of the one or more parameters for the at least one dental condition score for each dental user patient in at least one of: a visually interpretable form, or an electronic form.

11. The method of claim 1, further comprising:
(h) determining, by the one or more processing devices, at least one score comment corresponding to the at least one dental condition score for each dental user patient, the at least one score comment comprising at least one of: constructive non-medical feedback corresponding to the at least one dental condition score, or a non-medical affirmation corresponding to the at least one dental condition score; and
(i) producing the at least one score comment in at least one of: a visually interpretable form, or an electronic form, wherein at least one of: the constructive non-medical feedback corresponding to the at least one dental condition score, or the non-medical affirmation corresponding to the at least one dental condition score, comprises at least one of: a text message, an alpha-numeric message, or one or more symbols; and
wherein the at least one score comment comprises one or more of: a lifestyle suggestion, one or more learning materials, one or more dental care products, or one or more coaching suggestions.

12. The method of claim 1, wherein the selecting at least one machine-learning model further comprises:
(b-3) selecting a third machine-learning model based at least on the survey result information, the third machine-learning model corresponding to breath odor analysis; and
(b-4) selecting a fourth machine-learning model based at least on the survey result information, the fourth machine-learning model corresponding to at least one of: dentition/gum sensitivity analysis, enamel erosion analysis, dry mouth analysis, and/or mouth aging analysis.

13. A computer-implemented method, comprising:
(a) receiving, for each dental study subject of a plurality of dental study subjects, one or more dental treatment codes respectively relating to a dental history of each dental study subject;
(b) receiving, for each dental study subject of the plurality of dental study subjects, study survey result information respectively relating to an executed study survey by each dental study subject;
(c) selecting at least one machine learning model based on at least one of: the one or more dental treatment codes, or the study survey result information;
(d) associating, by the at least one machine learning model, the one or more dental treatment codes and the survey result information;
(e) calibrating the at least one machine-learning algorithm based, at least in part, on the associated one or more dental treatment codes and the received study survey result information;
(f) determining, by the at least one machine-learning model, a calibration dental condition score; and
(g) producing the at least one calibration dental condition score for each dental study subject in at least one of: a visually interpretable form, or an electronic form,
wherein at least steps (a)-(g) are performed by one or more processing devices.

14. The computer-implemented method of claim 13, wherein the calibrating the at least one machine-learning model further comprises at least one of:
(e-1) iteratively updating one or more parameters of the at least one machine-learning algorithm to minimize at least one of: an objective function, or a loss function, of the at least one machine-learning algorithm; or
(e-2) adjusting the one or more parameters of the at least one machine-learning model to reduce a deviation between the calibration dental condition score and the one or more dental treatment codes.

15. The computer-implemented method of claim 13, wherein the associating the one or more dental treatment codes and the survey result information further comprises:
(d-1) associating the one or more dental treatment codes respectively relating to a dental history of a respective dental study subject with the study survey result information relating to the executed study survey by the same respective dental study subject.

16. The computer-implemented method of claim 13, wherein the selecting the at least one machine learning model further comprises:
(c-1) determining a correspondence between the one or more dental treatment codes and at least one of: a first machine-learning algorithm model corresponding to gum disease analysis, or a second machine-learning model corresponding to teeth condition analysis; and
(c-2) selecting at least one of: the first machine-learning model, or the second machine-learning model, based on the determined correspondence;
(c-3) determining a correspondence between the one or more dental treatment codes and at least one of: a third machine-learning algorithm model corresponding to breath odor analysis, or a fourth machine-learning model corresponding to at least one of: dentition/gum sensitivity analysis, enamel erosion analysis, dry mouth analysis, and/or mouth aging analysis; and
(c-4) selecting at least one of: the third machine-learning model, or the fourth machine-learning model, based on the determined correspondence.

17. A computer-implemented method, comprising:
(a) receiving, for each dental study subject of the plurality of dental study subjects, study survey result information respectively relating to an executed study survey by each dental study subject;
(b) receiving, for one or more dental study subject of the plurality of dental study subjects, one or more dental records comprising dental records for treatment of dental caries and/or periodontitis;
(c) selecting at least one machine learning model based on at least one of: the study survey result information, or the one or more dental records;
(d) associating, by the at least one machine learning model, the one or more dental records and the survey result information;
(e) calibrating the at least one machine-learning algorithm based, at least in part, on the associated one or more dental records and the received study survey result information;
(f) determining, by the at least one machine-learning model, a calibration dental condition score; and
(g) producing the at least one calibration dental condition score for each dental study subject in at least one of: a visually interpretable form, or an electronic form,
wherein at least steps (a)-(g) are performed by one or more processing devices.

18. The computer-implemented method of claim 17, wherein the calibrating the at least one machine-learning model further comprises at least one of:
(e-1) iteratively updating one or more parameters of the at least one machine-learning algorithm to minimize at least one of: an objective function, or a loss function, of the at least one machine-learning algorithm; or
(e-2) adjusting the one or more parameters of the at least one machine-learning model to reduce a deviation between the calibration dental condition score and the one or more dental records.

19. The computer-implemented method of claim 17, wherein the associating the one or more dental records and the survey result information further comprises:
(d-1) associating the one or more dental records of the one or more respective dental study subjects with the study survey result information relating to the executed study survey by the same respective one or more dental study subjects.

20. The computer-implemented method of claim 17, wherein the selecting the at least one machine learning model further comprises:
(c-1) determining a correspondence between the one or more dental records and at least one of: a first machine-learning algorithm model corresponding to gum disease analysis, or a second machine-learning model corresponding to teeth condition analysis; and
(c-2) selecting at least one of: the first machine-learning model, or the second machine-learning model, based on the determined correspondence;
(c-3) determining a correspondence between the one or more dental records and at least one of: a third machine-learning algorithm model corresponding to breath odor analysis, or a fourth machine-learning model corresponding to at least one of: dentition/gum sensitivity analysis, enamel erosion analysis, dry mouth analysis, and/or mouth aging analysis; and
(c-4) selecting at least one of: the third machine-learning model, or the fourth machine-learning model, based on the determined correspondence.
